(19) United States
(12) Patent Application Publication
(10) Pub. No.: US 2014/0357576 A1
(43) Pub. Date: Dec. 4, 2014

(54) METHODS FOR ENHANCEMENT OF MUSCLE PROTEIN SYNTHESIS

(71) Applicant: Nestec S.A., Vevey (CH)

(72) Inventors: Denis BREUILLE, Lausanne (CH); Trent STELLINGWERFF, Victoria (CA); Daniel Ryan MOORE, Waterdown (CA); Elizabeth Ann OFFORD CAVIN, Montreux (CH); Stuart Martin PHILLIPS, Hamilton (CA)

(73) Assignee: Nestec S.A., Vevey (CH)

(21) Appl. No.: 14/272,642

(22) Filed: May 8, 2014

Related U.S. Application Data
(60) Provisional application No. 61/829,401, filed on May 31, 2013.
(60) Provisional application No. 61/834,731, filed on Jun. 13, 2013.
(60) Provisional application No. 61/863,013, filed on Aug. 7, 2013.

(51) Int. Cl. A61K 38/17; A61K 45/06; A23L 1/305; A61K 31/198

(52) U.S. Cl. CPC A61K 38/1709; A61K 31/198; A61K 45/06; A23L 1/3056; A23V 2002/00
USPC 514/21.2

(57) ABSTRACT

The present disclosure provides methods for enhancing muscle protein synthesis in an individual in need of same. Specifically, the present disclosure provides methods for enhancing muscle protein synthesis by enhancing myofibrillar muscle protein synthesis. The method includes administering to the individual a mixed macronutrient composition having (i) an amount of whey protein that is not capable of enhancing myofibrillar protein synthesis when ingested by itself and (ii) free leucine. The composition comprises at least about 5.0 g total leucine per dose.

### BACKGROUND

The present disclosure relates generally to health and nutrition. More specifically, the present disclosure relates to methods for enhancing muscle protein synthesis.

There are many types of nutritional compositions currently on the market. Nutritional compositions can be targeted toward certain consumer types, for example, young, elderly, athletic, etc., based on the specific ingredients of the nutritional composition. For example, provision of a complete mixture of amino acids has been shown to increase muscle protein synthesis rates through activation of the rapamycin complex. See, Bennet W M, Connacher A A, Scrimgeour C M, Smith K, Rennie M J. Increase in anterior tibialis muscle protein synthesis in healthy man during mixed amino acid infusion: studies of incorporation of [1-13C]leucine. Clin Sci (Lond) 1989; 76(4):447-54; and Dickinson J M, Fry C S, Drummond M J, et al. Mammalian target of rapamycin complex 1 activation is required for the stimulation of human skeletal muscle protein synthesis by essential amino acids. The Journal of Nutrition 2011; 141(5):856-62. This effect on muscle protein synthesis is primarily due to essential amino acids (“EAA”), as addition of non-essential amino acids does not stimulate increased muscle protein synthesis rates. See, Smith K, Reynolds N, Downie S, Patel A, Rennie M J. Effects of flooding amino acids on incorporation of labeled amino acids into human muscle protein. The American Journal of Physiology 1998; 275; and Tipton K D, Gurkin B E, Matin S, Wolfe R R. Nonessential amino acids are not necessary to stimulate net muscle protein synthesis in healthy volunteers. The Journal of nutritional biochemistry 1999; 10(2):89-95; and Volpi E, Kobayashi H, Sheffield-Moore M, Mittendorfer B, Wolfe R R. Essential amino acids are primarily responsible for the amino acid stimulation of muscle protein anabolism in healthy elderly adults. The American journal of clinical nutrition 2003; 78(2):250-8. Previous studies in humans have demonstrated a dose-dependent relationship between amino acid (see, Cuthbertson D, Smith K, Babraj J, et al. Anabolic signaling deficits underlie amino acid resistance of wasting, aging muscle. The FASEB journal: official publication of the Federation of American Societies for Experimental Biology 2005; 19(3):422-4) and protein intake (see, Moore D R, Robinson M J, Fry J L, et al. Ingested protein dose response of muscle and albumin protein synthesis after resistance exercise in young men. The American journal of clinical nutrition 2009; 89(1):161-8; and Yang Y, Breen L, Burd N A, et al. Resistance exercise enhances myofibrillar protein synthesis with graded intakes of whey protein in older men. The British Journal of Nutrition 2012:1-9) and muscle protein synthesis with ˜8.5 g EAA or ˜20 g protein sufficient to induce a maximal stimulation of muscle protein synthesis rates in young participants after exercise. The branched chain amino acid (“BCAA”) leucine appears to be a key determinant of the postprandial stimulation of muscle protein synthesis following protein intake. See, Norton L E, Layman D K, Bunpo P, Anthony T G, Brana D V, Garlick P J. The leucine content of a complete meal directs peak activation but not duration of skeletal muscle protein synthesis and mammalian target of rapamycin signaling in rats. The Journal of nutrition 2009; 139(6):1103-9. However, low doses of protein (e.g., about 6.0 or 6.25 g) have been found to be suboptimal for stimulating maximal muscle protein synthesis rates even within a mixed macronutrient beverage over an average 4.5 hours post-prandial period.

There exists a need, therefore, to provide individuals in need of muscle protein synthesis and, specifically, myofibrillar protein synthesis, with nutritional compositions that promote same.

### SUMMARY

The present disclosure is related to methods for enhancing muscle protein synthesis. Contrary to a majority of previous leucine studies, which measured mixed muscle protein synthesis (i.e., bulk proteins), the present disclosure is directed to measuring contractile myofibrillar protein specifically. Since these proteins are the functional proteins of the muscle, increasing synthesis of these proteins would presumably be a prerequisite to enhancing overall muscle strength. More specifically, myofibrillar proteins are force-generating proteins and represent about 60% of all muscle proteins. Therefore, if maintenance or increase of muscle mass and strength is desired, it is these specific proteins (not just all proteins indiscriminately, as mixed muscle protein synthesis would measure) that would require synthesis.

Accordingly, in a general embodiment, a method for enhancing muscle protein synthesis in an individual in need of same is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In another embodiment, a method for enhancing muscle anabolism in an individual in need of same is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In yet another embodiment, a method for enhancing muscle protein synthesis in an athlete is provided. The method includes administering to the athlete a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In still yet another embodiment, a method for enhancing muscle protein synthesis in an elderly individual is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In another embodiment, a method for enhancing muscle protein synthesis in an individual undergoing muscle rehabilitation is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In yet another embodiment, a method for enhancing muscle protein synthesis in an individual undergoing physical therapy is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In still yet another embodiment, a method for enhancing muscle protein synthesis in a young adult is provided. The method includes administering to the young adult a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In an embodiment, the composition is administered immediately following a resistance exercise.

In an embodiment, the composition is a liquid.

In an embodiment, the amount of whey protein is at least about 6.25 g per dose.

In an embodiment, the amount of free leucine is at least about 4.25 g per dose.

In an embodiment, the composition is administered immediately after resistance exercise.

In an embodiment, the mixed macronutrient includes a source of carbohydrates and a source of fats.

In an embodiment, the dose is a single bolus.

In an embodiment, the nutritional composition is formulated for administration to an individual selected from one of an infant, a child, a young adult, an elderly adult, an athlete, or combinations thereof.

In an embodiment, the administration occurs through an administration route selected from the group consisting of orally, a tube, a catheter, or combinations thereof.

In an embodiment, the composition further comprises a source of ω-3 fatty acids selected from the group consisting of fish oil, krill, plant sources containing ω-3 fatty acids, flaxseed, walnut, algae, or combinations thereof. In an embodiment, the ω-3 fatty acids are selected from the group consisting of α-linolenic acid (“ALA”), docosahexaenoic acid (“DHA”), eicosapentaenoic acid (“EPA”), or combinations thereof.

In an embodiment, the composition further comprises at least one nucleotide selected from the group consisting of a subunit of deoxyribonucleic acid (“DNA”), a subunit of ribonucleic acid (“RNA”), polymeric forms of DNA and RNA, yeast RNA, or combinations thereof. In an embodiment, the at least one nucleotide is an exogenous nucleotide.

In an embodiment, the composition further comprises a phytonutrient selected from the group consisting of flavanoids, allied phenolic compounds, polyphenolic compounds, terpenoids, alkaloids, sulphur-containing compounds, or combinations thereof.

In an embodiment, the phytonutrient is selected from the group consisting of carotenoids, plant sterols, quercetin, curcumin, limonin, or combinations thereof.

In an embodiment, the composition further comprises a prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

In an embodiment, the composition further comprises a probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

In an embodiment, the composition further comprises an amino acid selected from the group consisting of alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

In an embodiment, the composition further comprises an antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

In an embodiment, the composition further comprises a vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, or combinations thereof.

In an embodiment, the composition further comprises a mineral selected from the group consisting of boron, calcium, chromium, copper, iodine, iron, magnesium, manganese, molybdenum, nickel, phosphorus, potassium, selenium, silicon, tin, vanadium, zinc, or combinations thereof.

In an embodiment, the nutritional composition is an oral nutritional supplement. Alternatively, the nutritional composition may be a tube feeding.

In an embodiment, the nutritional composition is a source of complete nutrition. Alternatively, the nutritional composition may be a source of incomplete nutrition.

An advantage of the present disclosure is to provide methods for enhancing muscle protein synthesis.

Yet another advantage of the present disclosure is to provide methods for enhancing myofibrillar protein synthesis.

Another advantage of the present disclosure is to provide methods for enhancing muscle anabolism.

Yet another advantage of the present disclosure is to provide compositions having a low protein dose that are as effective at stimulating increased post-exercise myofibrillar protein synthesis as compositions having a high protein dose.

Another advantage of the present disclosure is to provide nutritional compositions that enhance immunity.

Still yet another advantage of the present disclosure is to provide methods for enhancing physical rehabilitation.

Yet another advantage of the present disclosure is to provide methods for muscle recovery after exercise.

Additional features and advantages are described herein, and will be apparent from the following Detailed Description and the figures.

### BRIEF DESCRIPTION OF THE FIGURES

FIG. 1 illustrates a schematic of an experimental protocol described herein below. Participants were block randomized to one of five possible treatment groups (see, Table 2) in a double-blind fashion (n=8 per treatment group). Treatments were administered immediately following the first set of bilateral skeletal muscle biopsies after unilateral resistance exercise (“RE”), consisting of 8 sets of 8-10 repetitions of seated knee extension with a 2-minute inter-set rest interval. Asterisk indicates blood sample; double upward arrow indicates bilateral biopsy.

FIG. 2 illustrates a mean (±SEM) plasma insulin concentration (μmol·L⁻¹) following treatment administration. Inset shows the AUC. Time course data were analyzed using a 2-factor (treatment×time) repeated measures ANOVA with Tukey's post hoc test (main effect for time, P<0.001). Times with different letters are significantly different from each other. AUC (inset) was analyzed using a 1-factor (treatment) ANOVA with Tukey's post hoc test (P=0.497). W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 3 illustrates a mean (±SEM) blood concentrations (μmol·L⁻¹) of leucine (A), isoleucine (B) valine (C) and ΣEAA (D) following treatment administration. No statistical analysis was performed on the time-course data. W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 4 illustrates a mean (±SEM) myofibrillar fractional synthetic rate (“FSR”) (%·h⁻¹) calculated during basal (“Fasted”) conditions, and over both early (0.0-1.5 hours), and late (1.5-4.5 hours) time periods during post-exercise recovery in both rested-fed (“FED”) (A) and exercise-fed (“EX-FED”) (B) conditions after treatment administration. Data were analyzed using a 3-factor (treatment×time×condition) mixed-model ANOVA with Tukey's post hoc test (treatment×time interaction, P=0.002). Times with different letters are statistically different from each other within that treatment. * Statistically different from W6+Low-Leu within that time; † statistically different from W6+BCAA within that time; ‡ statistically different from W6 within that time. W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 5 illustrates a mean (±SEM) intracellular free phenylalanine enrichments (tracer-to-tracee ratio—t·T⁻¹) from biopsies obtained at time 0 (“Fasted”), 1.5 hours, and 4.5 hours in both FED (A) and EX-FED (B) conditions. Conditions (FED and EX-FED) were analyzed separately using a 2-factor (treatment×time) repeated measures ANOVA with Tukey's post hoc test (FED: treatment, P=0.926; time, P=0.124; EX-FED: treatment, P=0.334; time, P=0.945). Conditions within each time were analyzed using a 2-factor (treatment×condition) ANOVA with Tukey's post hoc test (1.5 hours, P=0.055; 4.5 hours, P=0.317). Linear regression was used to examine the slope of intracellular free phenylalanine enrichment×time for each treatment in both FED (W6+Low-Leu: P=0.642; W6+BCAA: P=0.507; W6: P=0.911; W25: P=0.136; W6+High-Leu: P=0.914) and EX-FED (W6+Low-Leu: P=0.244; W6+BCAA: P=0.777; W6: P=0.934; W25: P=0.422; W6+High-Leu: P=0.438) conditions. W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 6 illustrates a mean (±SEM) plasma free phenylalanine enrichments (tracer-to-tracee ratio t·T⁻¹) over time. Time course data were analyzed using a 2-factor (treatment×time) repeated measures ANOVA with Tukey's post hoc test (treatment, P=0.917; time, P=0.58). Linear regression was used to examine the slope of plasma free phenylalanine enrichment×time for each treatment (W6+Low-Leu: P=0.858; W6+BCAA: P=0.357; W6: P=0.455; W25: P=0.424; W6+High-Leu: P=0.156). W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 7 illustrates a representative western blot images for p-Akt^{Ser473}, p-mToR^{Ser2448}, p-p70S6k^{Thr389}, p-4E-BP1^{Thr 37/46}, p-rpS6^{Ser240/244}, p-eEF2^{Thr56}, and α-tubulin during Fasted, and 1.5 hours EX-FED, 1.5 hours FED, 4.5 hours EX-FED; and 4.5 hours FED following nutrient treatment administration. W6+Low-Leu (6.25 g whey protein supplemented with leucine, for 3.0 g total leucine); W6+BCAA (6.25 g whey protein supplemented with leucine, isoleucine, and valine, for 5.0 g total leucine); W6 (6.25 g whey protein); W25 (25 g whey protein); W6+High-Leu (6.25 g whey protein supplemented with leucine, for 5.0 g total leucine).

FIG. 8 illustrates the effect of ingested protein-leucine quantity on the myofibrillar FSR during the first 240-min of recovery from cycling. Data are means and SD.

FIG. 9 illustrates the effect of ingested protein-leucine quantity on (A) plasma leucine, (B) essential, and (C) total amino acid concentrations during the first 240-minutes of recovery from cycling. Data are means and SD.

FIG. 10 illustrates the effect of ingested protein-leucine quantity on phosphorylation of (A) mTOR, (B) p70S6K, (C) 4E-BP1, and (D) rpS6 during recovery from cycling. Data are means and SD.

FIG. 11 illustrates the association between myofibrillar FSR and (A) mean plasma leucine concentration between 0-240 minutes of recovery, (B) ratio of 4E-BP1γ to total 4E-BP1 phosphorylation, and (C) rpS6 phosphorylation at 240 minutes of recovery.

FIG. 12 illustrates the top ranked networks arising from the interrogation of the transcriptome responding to protein-leucine quantity at 30 minutes (plates A-C) and 240 minutes (plates D-F) into recovery. Selected molecular function modules (colored shading and labeling) are representative of the top-ranked within-network biology. Symbol color indicates the direction of gene regulation: green, down-regulated; red, up-regulated; grey, within global gene selection but not significantly affected within contrast. Gene symbols denoted with an asterisk represent multiple microarray probe selections.

FIG. 13 illustrates the correlation between mean differential gene expression estimated from the Illumina microarray vs Nanostring nCounter.

FIG. 14 illustrates a summary of the effect of protein-leucine feeding on modulation the molecular programme regulating skeletal muscle regeneration from intense endurance exercise.

### DETAILED DESCRIPTION

All dosage ranges contained within this application are intended to include all numbers, whole or fractions, contained within said range.

As used in this disclosure and the appended claims, the singular forms “a,” “an” and “the” include plural referents unless the context clearly dictates otherwise. Thus, for example, reference to “a polypeptide” includes a mixture of two or more polypeptides, and the like.

As used herein, “about” is understood to refer to numbers in a range of numerals. Moreover, all numerical ranges herein should be understood to include all integer, whole or fractions, within the range.

As used herein the term “amino acid” is understood to include one or more amino acids. The amino acid can be, for example, alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

As used herein, “animal” includes, but is not limited to, mammals, which include but is not limited to, rodents, aquatic mammals, domestic animals such as dogs and cats, farm animals such as sheep, pigs, cows and horses, and humans. Wherein the terms “animal” or “mammal” or their plurals are used, it is contemplated that it also applies to any animals that are capable of the effect exhibited or intended to be exhibited by the context of the passage.

As used herein, the term “antioxidant” is understood to include any one or more of various substances such as beta-carotene (a vitamin A precursor), vitamin C, vitamin E, and selenium) that inhibit oxidation or reactions promoted by Reactive Oxygen Species (“ROS”) and other radical and non-radical species. Additionally, antioxidants are molecules capable of slowing or preventing the oxidation of other molecules. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

As used herein, “complete nutrition” includes nutritional products and compositions that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is an amount that prevents a deficiency, treats a disease or medical condition in an individual or, more generally, reduces symptoms, manages progression of the diseases or provides a nutritional, physiological, or medical benefit to the individual. A treatment can be patient- or doctor-related.

While the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, sources of ω-3 fatty acids include, for example, fish oil, krill, plant sources of ω-3, flaxseed, walnut, and algae. Examples of ω-3 fatty acids include, for example, α-linolenic acid (“ALA”), docosahexaenoic acid (“DHA”), eicosapentaenoic acid (“EPA”), or combinations thereof.

As used herein, “food grade micro-organisms” means micro-organisms that are used and generally regarded as safe for use in food.

As used herein, “incomplete nutrition” includes nutritional products or compositions that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “long term administrations” are preferably continuous administrations for more than 6 weeks. Alternatively, “short term administrations,” as used herein, are continuous administrations for less than 6 weeks.

As used herein, “mammal” includes, but is not limited to, rodents, aquatic mammals, domestic animals such as dogs and cats, farm animals such as sheep, pigs, cows and horses, and humans. Wherein the term “mammal” is used, it is contemplated that it also applies to other animals that are capable of the effect exhibited or intended to be exhibited by the mammal.

The term “microorganism” is meant to include the bacterium, yeast and/or fungi, a cell growth medium with the microorganism, or a cell growth medium in which microorganism was cultivated.

As used herein, the term “minerals” is understood to include boron, calcium, chromium, copper, iodine, iron, magnesium, manganese, molybdenum, nickel, phosphorus, potassium, selenium, silicon, tin, vanadium, zinc, or combinations thereof.

As used herein, a “non-replicating” microorganism means that no viable cells and/or colony forming units can be detected by classical plating methods. Such classical plating methods are summarized in the microbiology book: James Monroe Jay, et al., Modern food microbiology, 7th edition, Springer Science, New York, N.Y. p. 790 (2005). Typically, the absence of viable cells can be shown as follows: no visible colony on agar plates or no increasing turbidity in liquid growth medium after inoculation with different concentrations of bacterial preparations (′non replicating′ samples) and incubation under appropriate conditions (aerobic and/or anaerobic atmosphere for at least 24 h). For example, bifidobacteria such as Bifidobacterium longum, Bifidobacterium lactis and Bifidobacterium breve or lactobacilli, such as Lactobacillus paracasei or Lactobacillus rhamnosus, may be rendered non-replicating by heat treatment, in particular low temperature/long time heat treatment.

As used herein, a “nucleotide” is understood to be a subunit of deoxyribonucleic acid (“DNA”), ribonucleic acid (“RNA”), polymeric RNA, polymeric DNA, or combinations thereof. It is an organic compound made up of a nitrogenous base, a phosphate molecule, and a sugar molecule (deoxyribose in DNA and ribose in RNA). Individual nucleotide monomers (single units) are linked together to form polymers, or long chains. Exogenous nucleotides are specifically provided by dietary supplementation. The exogenous nucleotide can be in a monomeric form such as, for example, 5′-Adenosine Monophosphate (“5′-AMP”), 5′-Guanosine Monophosphate (“5′-GMP”), 5′-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”), or combinations thereof. The exogenous nucleotide can also be in a polymeric form such as, for example, an intact RNA. There can be multiple sources of the polymeric form such as, for example, yeast RNA.

“Nutritional products,” or “nutritional compositions,” as used herein, are understood to include any number of optional additional ingredients, including conventional food additives (synthetic or natural), for example one or more acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount. The nutritional products or compositions may be a source of complete nutrition or may be a source of incomplete nutrition.

As used herein the term “patient” is understood to include an animal, especially a mammal, and more especially a human that is receiving or intended to receive treatment, as it is herein defined.

As used herein, “phytochemicals” or “phytonutrients” are non-nutritive compounds that are found in many foods. Phytochemicals are functional foods that have health benefits beyond basic nutrition, are health promoting compounds that come from plant sources, and may be natural or purified. “Phytochemicals” and “Phytonutrients” refers to any chemical produced by a plant that imparts one or more health benefit on the user. Non-limiting examples of phytochemicals and phytonutrients include those that are:

i) phenolic compounds which include monophenols (such as, for example, apiole, carnosol, carvacrol, dillapiole, rosemarinol); flavonoids (polyphenols) including flavonols (such as, for example, quercetin, fingerol, kaempferol, myricetin, rutin, isorhamnetin), flavanones (such as, for example, fesperidin, naringenin, silybin, eriodictyol), flavones (such as, for example, apigenin, tangeritin, luteolin), flavan-3-ols (such as, for example, catechins, (+)-catechin, (+)-gallocatechin, (−)-epicatechin, (−)-epigallocatechin, (−)-epigallocatechin gallate (EGCG), (−)-epicatechin 3-gallate, theaflavin, theaflavin-3-gallate, theaflavin-3′-gallate, theaflavin-3,3′-digallate, thearubigins), anthocyanins (flavonals) and anthocyanidins (such as, for example, pelargonidin, peonidin, cyanidin, delphinidin, malvidin, petunidin), isoflavones (phytoestrogens) (such as, for example, daidzein (formononetin), genistein (biochanin A), glycitein), dihydroflavonols, chalcones, coumestans (phytoestrogens), and Coumestrol; Phenolic acids (such as: Ellagic acid, Gallic acid, Tannic acid, Vanillin, curcumin); hydroxycinnamic acids (such as, for example, caffeic acid, chlorogenic acid, cinnamic acid, ferulic acid, coumarin); lignans (phytoestrogens), silymarin, secoisolariciresinol, pinoresinol and lariciresinol); tyrosol esters (such as, for example, tyrosol, hydroxytyrosol, oleocanthal, oleuropein); stilbenoids (such as, for example, resveratrol, pterostilbene, piceatannol) and punicalagins;

ii) terpenes (isoprenoids) which include carotenoids (tetraterpenoids) including carotenes (such as, for example, α-carotene, β-carotene, γ-carotene, δ-carotene, lycopene, neurosporene, phytofluene, phytoene), and xanthophylls (such as, for example, canthaxanthin, cryptoxanthin, aeaxanthin, astaxanthin, lutein, rubixanthin); monoterpenes (such as, for example, limonene, perillyl alcohol); saponins; lipids including: phytosterols (such as, for example, campesterol, beta sitosterol, gamma sitosterol, stigmasterol), tocopherols (vitamin E), and omega-3, 6, and 9 fatty acids (such as, for example, gamma-linolenic acid); triterpenoid (such as, for example, oleanolic acid, ursolic acid, betulinic acid, moronic acid);

iii) betalains which include Betacyanins (such as: betanin, isobetanin, probetanin, neobetanin); and betaxanthins (non glycosidic versions) (such as, for example, indicaxanthin, and vulgaxanthin);

iv) organosulfides, which include, for example, dithiolthiones (isothiocyanates) (such as, for example, sulphoraphane); and thiosulphonates (allium compounds) (such as, for example, allyl methyl trisulfide, and diallyl sulfide), indoles, glucosinolates, which include, for example, indole-3-carbinol; sulforaphane; 3,3′-diindolylmethane; sinigrin; allicin; alliin; allyl isothiocyanate; piperine; syn-propanethial-S-oxide;

v) protein inhibitors, which include, for example, protease inhibitors;

vi) other organic acids which include oxalic acid, phytic acid (inositol hexaphosphate); tartaric acid; and anacardic acid; or

vii) combinations thereof.

As used herein, a “prebiotic” is a food substance that selectively promotes the growth of beneficial bacteria or inhibits the growth or mucosal adhesion of pathogenic bacteria in the intestines. They are not inactivated in the stomach and/or upper intestine or absorbed in the gastrointestinal tract of the person ingesting them, but they are fermented by the gastrointestinal microflora and/or by probiotics. Prebiotics are, for example, defined by Glenn R. Gibson and Marcel B. Roberfroid, “Dietary Modulation of the Human Colonic Microbiota: Introducing the Concept of Prebiotics,” J. Nutr., 125: 1401-1412 (1995). Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof.

As used herein, probiotic micro-organisms (hereinafter “probiotics”) are food-grade microorganisms (alive, including semi-viable or weakened, and/or non-replicating), metabolites, microbial cell preparations or components of microbial cells that could confer health benefits on the host when administered in adequate amounts, more specifically, that beneficially affect a host by improving its intestinal microbial balance, leading to effects on the health or well-being of the host. See, Salminen S, Ouwehand A. Benno Y. et al., “Probiotics: how should they be defined?,” Trends Food Sci. Technol., 10, 107-10 (1999). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, or combinations thereof.

The terms “protein,” “peptide,” “oligopeptides” or “polypeptide,” as used herein, are understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combinations thereof. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to, acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycation, glycosylation, glycosylphosphatidyl inositol (“GPI”) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide.

Non-limiting examples of proteins include dairy based proteins, plant based proteins, animal based proteins and artificial proteins. Dairy based proteins may be selected from the group consisting of casein, caseinates, casein hydrolysate, whey, whey hydrolysates, whey concentrates, whey isolates, milk protein concentrate, milk protein isolate, or combinations thereof. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, buckwheat, lentils, pulses, single cell proteins, or combinations thereof. Animal based proteins may be selected from the group consisting of beef, poultry, fish, lamb, seafood, or combinations thereof.

As used herein, the term “rehabilitation” refers to the process of restoring an individual to good, physical condition, operation, or capacity after decrease of same. Accordingly, rehabilitation may include physical therapy, exercise or the like. Examples of individuals in need of rehabilitation include, but are not limited to, individuals having had muscle losses due to immobilization/bed rest, individuals in the hospital, individuals recovering from a critical illness or acute disease, individuals suffering from physical limitations, elderly individuals, athletes, infants experiencing growth retardation, etc.

As used herein, the terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition, such as nitrogen imbalance or muscle loss. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition.

As used herein, a “tube feed” is a complete or incomplete nutritional product or composition that is administered to an animal's gastrointestinal system, other than through oral administration, including but not limited to a nasogastric tube, orogastric tube, gastric tube, jejunostomy tube (“J-tube”), percutaneous endoscopic gastrostomy (“PEG”), port, such as a chest wall port that provides access to the stomach, jejunum and other suitable access ports.

As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e. MK-4, MK-7), folic acid and biotin) essential in minute amounts for normal growth and activity of the body and obtained naturally from plant and animal foods or synthetically made, pro-vitamins, derivatives, analogs.

The present disclosure is related to methods for enhancing muscle protein synthesis. Specifically, the present disclosure is related to methods for enhancing myofibrillar-protein fractional synthesis rates by luecine supplementation of a low protein mixed macronutrient food product (e.g., a beverage). Provision of a complete mixture of amino acids has been shown to increase muscle protein synthesis rates through activation of the rapamycin complex. See, Bennet W M, Connacher A A, Scrimgeour C M, Smith K, Rennie M J. Increase in anterior tibialis muscle protein synthesis in healthy man during mixed amino acid infusion: studies of incorporation of [1-13C]leucine. Clin Sci (Lond) 1989; 76(4):447-54; and Dickinson J M, Fry C S, Drummond M J, et al. Mammalian target of rapamycin complex 1 activation is required for the stimulation of human skeletal muscle protein synthesis by essential amino acids. The Journal of Nutrition 2011; 141(5):856-62. This effect on muscle protein synthesis is primarily due to essential amino acids (“EAA”), as addition of non-essential amino acids does not stimulate increased muscle protein synthesis rates. See, Smith K, Reynolds N, Downie S, Patel A, Rennie M J. Effects of flooding amino acids on incorporation of labeled amino acids into human muscle protein. The American Journal of Physiology 1998; 275; and Tipton K D, Gurkin B E, Matin S, Wolfe R R. Nonessential amino acids are not necessary to stimulate net muscle protein synthesis in healthy volunteers. The Journal of nutritional biochemistry 1999; 10(2):89-95; and Volpi E, Kobayashi H, Sheffield-Moore M, Mittendorfer B, Wolfe R R. Essential amino acids are primarily responsible for the amino acid stimulation of muscle protein anabolism in healthy elderly adults. The American journal of clinical nutrition 2003; 78(2):250-8. Previous studies in humans have demonstrated a dose-dependent relationship between amino acid (see, Cuthbertson D, Smith K, Babraj J, et al. Anabolic signaling deficits underlie amino acid resistance of wasting, aging muscle. The FASEB journal: official publication of the Federation of American Societies for Experimental Biology 2005; 19(3):422-4) and protein intake (see, Moore D R, Robinson M J, Fry J L, et al. Ingested protein dose response of muscle and albumin protein synthesis after resistance exercise in young men. The American journal of clinical nutrition 2009; 89(1):161-8; and Yang Y, Breen L, Burd N A, et al. Resistance exercise enhances myofibrillar protein synthesis with graded intakes of whey protein in older men. The British Journal of Nutrition 2012:1-9) and muscle protein synthesis with ˜8.5 g EAA or ˜20 g protein sufficient to induce a maximal stimulation of muscle protein synthesis rates in young participants after exercise. The branched chain amino acid (“BCAA”) leucine appears to be a key determinant of the postprandial stimulation of muscle protein synthesis following protein intake. See, Norton L E, Layman D K, Bunpo P, Anthony T G, Brana D V, Garlick P J. The leucine content of a complete meal directs peak activation but not duration of skeletal muscle protein synthesis and mammalian target of rapamycin signaling in rats. The Journal of nutrition 2009; 139(6):1103-9. However, low doses of protein (e.g., about 6.0 or 6.25 g) have been found to be suboptimal for stimulating maximal muscle protein synthesis rates even within a mixed macronutrient beverage over an average 4.5 hours post-prandial period.

In-vivo animal based studies have shown that the independent administration of leucine, but not isoleucine or valine, can stimulate muscle protein synthesis rates to the same extent as a complete mixture of amino acids or complete protein. See, Anthony J C, Yoshizawa F, Anthony T G, Vary T C, Jefferson L S, Kimball S R. Leucine stimulates translation initiation in skeletal muscle of postabsorptive rats via a rapamycin-sensitive pathway. The Journal of nutrition 2000; 130(10):2413-9; and Escobar J, Frank J W, Suryawan A, et al. Regulation of cardiac and skeletal muscle protein synthesis by individual branched-chain amino acids in neonatal pigs. American journal of physiology Endocrinology and metabolism 2006; 290(4); and Crozier S J, Kimball S R, Emmert S W, Anthony J C, Jefferson L S. Oral leucine administration stimulates protein synthesis in rat skeletal muscle. The Journal of nutrition 2005; 135(3):376-82; and Anthony J C, Lang C H, Crozier S J, et al. Contribution of insulin to the translational control of protein synthesis in skeletal muscle by leucine. American journal of physiology Endocrinology and metabolism 2002; 282(5):E1092-101. Given these findings, research in humans has focused on the efficacy of leucine supplementation to promote increases in muscle protein synthesis and lean mass accretion. See, Churchward-Venne T A, Burd N A, Mitchell C J, et al. Supplementation of a suboptimal protein dose with leucine or essential amino acids: effects on myofibrillar protein synthesis at rest and following resistance exercise in men. The Journal of physiology 2012; and Koopman R, Verdijk L, Manders R J, et al. Co-ingestion of protein and leucine stimulates muscle protein synthesis rates to the same extent in young and elderly lean men. The American journal of clinical nutrition 2006; 84(3):623-32; and Koopman R, Wagenmakers A J, Manders R J, et al. Combined ingestion of protein and free leucine with carbohydrate increases postexercise muscle protein synthesis in vivo in male subjects. American journal of physiology Endocrinology and metabolism 2005; 288(4):E645-53; and Tipton K D, Elliott T A, Ferrando A A, Aarsland A A, Wolfe R R. Stimulation of muscle anabolism by resistance exercise and ingestion of leucine plus protein. Applied physiology, nutrition, and metabolism=Physiologie appliquee, nutrition et metabolisme 2009; 34(2):151-61; and Verhoeven S, Vanschoonbeek K, Verdijk L B, et al. Long-term leucine supplementation does not increase muscle mass or strength in healthy elderly men. The American journal of clinical nutrition 2009; 89(5):1468-75; and Leenders M, Verdijk L B, van der Hoeven L, et al. Prolonged leucine supplementation does not augment muscle mass or affect glycemic control in elderly type 2 diabetic men. The Journal of nutrition 2011; 141(6):1070-6.

While some human studies have demonstrated increased rates of muscle protein synthesis following leucine administration (see, Smith K, Barua J M, Watt P W, Scrimgeour C M, Rennie M J. Flooding with L-[1-13C]leucine stimulates human muscle protein incorporation of continuously infused L-[1-13C]valine. The American journal of physiology 1992; 262(3 Pt 1):E372-6; and Rieu I, Balage M, Sornet C, et al. Leucine supplementation improves muscle protein synthesis in elderly men independently of hyperaminoacidaemia. The Journal of physiology 2006; 575(Pt 1):305-15; and Katsanos C S, Kobayashi H, Sheffield-Moore M, Aarsland A, Wolfe R R. A high proportion of leucine is required for optimal stimulation of the rate of muscle protein synthesis by essential amino acids in the elderly. American journal of physiology Endocrinology and metabolism 2006; 291(2):E381-7), others have not (see, Tipton K D, Elliott T A, Ferrando A A, Aarsland A A, Wolfe R R. Stimulation of muscle anabolism by resistance exercise and ingestion of leucine plus protein. Applied physiology, nutrition, and metabolism=Physiologie appliquee, nutrition et metabolisme 2009; 34(2):151-61; and Nair K S, Schwartz R G, Welle S. Leucine as a regulator of whole body and skeletal muscle protein metabolism in humans. The American journal of physiology 1992; 263(5 Pt 1):E928-34; and Glynn E L, Fry C S, Drummond M J, et al. Excess leucine intake enhances muscle anabolic signaling but not net protein anabolism in young men and women. The Journal of nutrition 2010; 140(11):1970-6; and Koopman R, Verdijk L B, Beelen M, et al. Co-ingestion of leucine with protein does not further augment post-exercise muscle protein synthesis rates in elderly men. The British journal of nutrition 2008; 99(3):571-80); however, provision of leucine has been reported to result in reduced circulating concentrations of both isoleucine and valine (see, Verhoeven S, Vanschoonbeek K, Verdijk L B, et al. Long-term leucine supplementation does not increase muscle mass or strength in healthy elderly men. The American journal of clinical nutrition 2009; 89(5):1468-75; and wendseid M E, Villalobos J, Figueroa W S, Drenick E J. The effects of test doses of leucine, isoleucine or valine on plasma amino acid levels. The unique effect of leucine. The American journal of clinical nutrition 1965; 17(5):317-21; and Hagenfeldt L, Eriksson S, Wahren J. Influence of leucine on arterial concentrations and regional exchange of amino acids in healthy subjects. Clin Sci (Lond) 1980; 59(3):173-81), the decline of which have been shown to coincide with reduced muscle protein synthesis rates in animals (see, Escobar J, Frank J W, Suryawan A, Nguyen H V, Davis T A. Amino acid availability and age affect the leucine stimulation of protein synthesis and eIF4F formation in muscle. American journal of physiology Endocrinology and metabolism 2007; 293(6):E1615-21). Whether a decrease in circulating concentrations of isoleucine and/or valine would reduce the magnitude or duration of muscle protein synthesis rates in humans is unknown.

In Example 1 of the present disclosure, Applicant performed experiments to assess the impact of supplementing a low dose of protein (6.25 g whey), previously demonstrated to be less than maximally effective in stimulating muscle protein synthesis (through the stimulating of myofibrillar protein synthesis) following exercise (see, Moore D R, Robinson M J, Fry J L, et al. Ingested protein dose response of muscle and albumin protein synthesis after resistance exercise in young men. The American journal of clinical nutrition 2009; 89(1):161-8), within a mixed macronutrient beverage with: (i) a lower dose of leucine (W6+Low-Leu, 3.0 g leucine); (ii) a higher dose of leucine (W6+High-Leu, 5.0 g leucine), and (iii) a higher dose of leucine, with additional isoleucine, and valine (W6+BCAA, 5.0 g leucine plus valine and isoleucine) on myofibrillar protein synthesis rates and the phosphorylation of protein targets of the Akt-mTORC1 pathway both ‘early’ (0-1.5 hours) and ‘late’ (1.5-4.5 hours) at rest and during post-exercise recovery.

Indeed, as discussed above, the present disclosure is directed to measuring contractile myofibrillar protein specifically. Since these proteins are the functional proteins of the muscle, increasing synthesis of these proteins would presumably be a prerequisite to enhancing overall muscle strength. More specifically, myofibrillar proteins are force-generating proteins and represent about 60% of all muscle proteins. Therefore, if maintenance or increase of muscle mass and strength is desired, it is these specific proteins (not just all proteins indiscriminately, as mixed muscle protein synthesis would measure) that would require synthesis.

Accordingly, in the experiments described herein below, Applicant investigated the effects on myofibrillar protein synthesis from ingestion of a macronutrient beverage with varying doses of leucine. Specifically, Applicant examined a temporally early and late period after treatment since leucine has been suggested to direct the peak activation, but not duration of the myofibrillar protein synthesis response (see, Norton L E, Layman D K, Bunpo P, Anthony T G, Brana D V, Garlick P J. The leucine content of a complete meal directs peak activation but not duration of skeletal muscle protein synthesis and mammalian target of rapamycin signaling in rats. The Journal of nutrition 2009; 139(6):1103-9). Applicant used, as positive and negative controls, 25 g of whey protein (“W25”, 3.0 g leucine), a dose of protein sufficient to induce a maximal stimulation of myofibrillar protein synthesis rates after resistance exercise and a lower sub-optimal dose (6.25 g) of whey protein (“W6”, 0.75 g leucine). See, Moore D R, Robinson M J, Fry J L, et al. Ingested protein dose response of muscle and albumin protein synthesis after resistance exercise in young men. The American journal of clinical nutrition 2009; 89(1):161-8. Applicant hypothesized that W6+Low-Leu, W6+BCAA, W25, and W6+High-Leu would stimulate greater postprandial myofibrillar protein synthesis rates than W6 under resting conditions with no differences between treatments. During post-exercise recovery, Applicant hypothesized that W6+BCAA, W25, and W6+High-Leu would elicit similar increases in myofibrillar protein synthesis, but greater than W6+Low-Leu and W6 due to maintenance of the myofibrillar protein synthesis response a more sustained response over the later periods examined.

As discussed below in Example 1, Applicant was able to demonstrate that the addition of a higher dose of leucine to a mixed macronutrient beverage containing ˜33% of a previously demonstrated suboptimal protein dose can enhance myofibrillar protein synthesis to same level as that seen with four times as much whey protein. In other words, provision of leucine to ‘trigger’ myofibrillar protein synthesis rates along with a small dose of protein to provide essential and non-essential amino acid substrate within a mixed macronutrient beverage was shown to be an effective strategy to stimulate increased myofibrillar protein synthesis rates.

Muscle protein synthesis is increased in response to exercise and protein feeding in healthy individuals (see, Rennie M J, Wackerhage H, Spangenburg E E, Booth F W. Control of the size of the human muscle mass. Annual review of physiology 2004; 66:799-828), and is the primary variable determining changes in net protein balance (see, Glynn E L, Fry C S, Drummond M J, et al. Muscle protein breakdown has a minor role in the protein anabolic response to essential amino acid and carbohydrate intake following resistance exercise. American journal of physiology Regulatory, integrative and comparative physiology 2010; 299(2):R533-40). It has been demonstrated that muscle protein synthesis is stimulated in a protein/EAA dose-dependent manner up to ˜10 g of EAA at rest (see, Cuthbertson D, Smith K, Babraj J, et al. Anabolic signaling deficits underlie amino acid resistance of wasting, aging muscle. The FASEB journal: official publication of the Federation of American Societies for Experimental Biology 2005; 19(3):422-4), and ˜20 g of protein (˜8.6 g EAA) after resistance exercise (see, Moore D R, Robinson M J, Fry J L, et al. Ingested protein dose response of muscle and albumin protein synthesis after resistance exercise in young men. The American journal of clinical nutrition 2009; 89(1):161-8). In Example 1 of the present disclosure, Applicant utilized whey protein as the base protein source because it has demonstrated efficacy as a high quality protein source able to robustly stimulate myofibrillar protein synthesis rates (see, Churchward-Venne T A, Burd N A, Phillips S M, Research Group EM. Nutritional regulation of muscle protein synthesis with resistance exercise: strategies to enhance anabolism. Nutrition & metabolism 2012; 9(1):40). However, protein is typically co-ingested with CHO and fat with a meal which would alter the kinetics of gut amino acid absorption (see, Burke L M, Winter J A, Cameron-Smith D, Enslen M, Farnfield M, Decombaz J. Effect of intake of different dietary protein sources on plasma amino Acid profiles at rest and after exercise. International journal of sport nutrition and exercise metabolism 2012; 22(6):452-62). Thus, in Example 1 described below, Applicant tested the efficacy of mixed macronutrient drinks with varying doses of whey to stimulate myofibrillar protein synthesis.

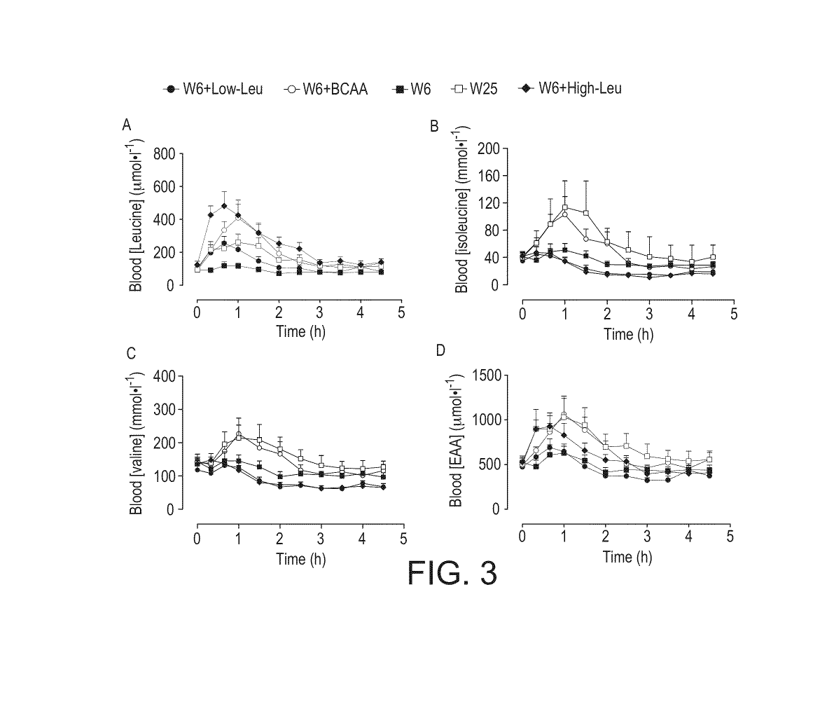

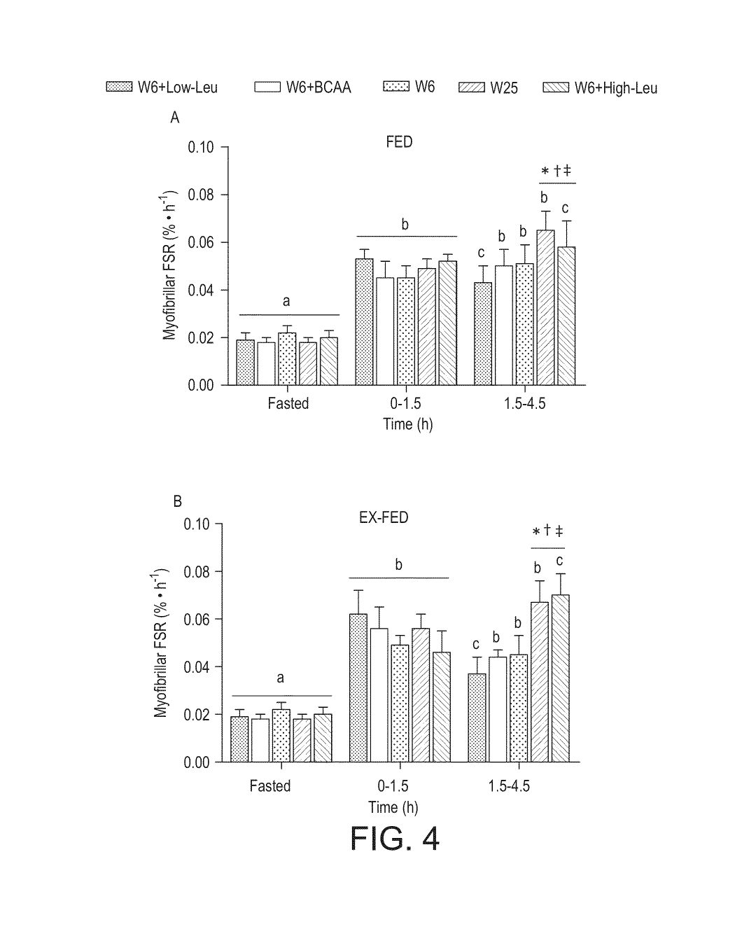

Consistent with Applicant's previous results utilizing protein-only feeding (see, Moore D R, Robinson M J, Fry J L, et al. Ingested protein dose response of muscle and albumin protein synthesis after resistance exercise in young men. The American journal of clinical nutrition 2009; 89(1):161-8), in this study Applicant found that a low dose of protein (W6) was suboptimal for stimulating maximal myofibrillar protein synthesis rates even within a mixed macronutrient beverage over an average 4.5 hours post-prandial period. Interestingly, supplementing this low protein dose with a high proportion of leucine (W6+High-Leu) stimulated myofibrillar protein synthesis to an equivalent magnitude and duration to that stimulated following ingestion of an energy-matched mixed macronutrient beverage containing 25 g of whey protein (W25), which would, based on past data, be a dose of protein and EAA sufficient to induce a maximal stimulation of myofibrillar protein synthesis rates at rest and following resistance exercise. While there were no differences among treatments in myofibrillar protein synthesis during the ‘early’ 0-1.5 hours post-exercise period, W6+High-Leu and W25 stimulated greater myofibrillar protein synthesis over the ‘late’ 1.5-4.5 hours period than each of the other treatments (see, FIG. 4). The somewhat surprising lack of difference in myofibrillar protein synthesis rates among treatments during the ‘early’ post-exercise/feeding period occurred despite quite divergent blood and intramuscular leucine, isoleucine, valine, and EAA concentrations (see, FIG. 3 and Table 3). Presumably, this lack of difference early after feeding (i.e., up to 1.5 hours) suggests that amino acid availability and/or nutrient signals (leucine) that play a role in regulating myofibrillar protein synthesis were equivalently stimulated in all conditions even by the small amount of leucine in the W6 treatment. In contrast, in the latter portion of the protocol only in the W6+High-Leu treatment was an myofibrillar protein synthesis response elicited that was not different from that of W25 despite containing only one quarter of the protein dose and ˜62% of the EAA content. That the W6+High-Leu was so effective may relate to the fact that this treatment elicited the greatest blood leucine AUC (see, Table 3), the greatest intracellular leucine concentration when assessed at 1.5 hours (see, Table 4), and a sustained increase in the phosphorylation of mTOR^{(ser2448)} at 4.5 hours (see, Table 5). Applicant also observed an increase in the phosphorylation of targets downstream of mToR^{(ser2448)} including 4E-BP1^{(Thr37/46)} and rpS6^{(Ser 240.244)} although there were no statistically significant treatment dependent differences (see, Table 5). In partial agreement with Applicant's observation of the potency of the W6+High-Leu treatment, previous studies have shown that a high proportion of leucine (3.5 vs. 1.8 g) within a 10 g EAA solution resulted in greater intramuscular cell signaling and a more prolonged mixed myofibrillar protein synthesis response (see, Glynn E L, Fry C S, Drummond M J, et al. Excess leucine intake enhances muscle anabolic signaling but not net protein anabolism in young men and women. The Journal of nutrition 2010; 140(11):1970-6).

Applicant's previous studies have shown that feeding 25 g of whey protein results in increased myofibrillar protein synthesis rates over 1-3 hours that are not enhanced by prior resistance exercise (see, Churchward-Venne T A, Burd N A, Mitchell C J, et al. Supplementation of a suboptimal protein dose with leucine or essential amino acids: effects on myofibrillar protein synthesis at rest and following resistance exercise in men. The Journal of physiology 2012; and Moore D R, Tang J E, Burd N A, Rerecich T, Tarnopolsky M A, Phillips S M. Differential stimulation of myofibrillar and sarcoplasmic protein synthesis with protein ingestion at rest and after resistance exercise. The Journal of physiology 2009; 587(Pt 4):897-904); however, while myofibrillar protein synthesis rates return to basal levels over 3-5 hours under resting conditions, the augmented rates of myofibrillar protein synthesis with resistance exercise and feeding are generally observed over this later period in recovery. Applicant has previously shown that a suboptimal protein dose (6.25 g whey) supplemented with leucine (total leucine=3.0 g) or a complete mixture of EAA devoid of leucine (total leucine=0.75 g) can stimulate postprandial myofibrillar protein synthesis rates equivalent to that stimulated following ingestion of 25 g whey protein (total leucine=3.0 g) under resting but not post-resistance exercise conditions (see, Churchward-Venne T A, Burd N A, Mitchell C J, et al. Supplementation of a suboptimal protein dose with leucine or essential amino acids: effects on myofibrillar protein synthesis at rest and following resistance exercise in men. The Journal of physiology 2012).

Similarly, in Example 1, W6+Low-Leu (total leucine=3.0 g) and W6 (total leucine=0.75 g) were as effective as W25 (total leucine=3.0 g) at stimulating myofibrillar protein synthesis rates when assessed during the early 0-1.5 hours, but not the later 1.5-4.5 hours period. Applicant found no difference between FED vs. EX-FED myofibrillar protein synthesis rates, which could be due to the choice of tissue sampling times.

The experimental results disclosed in Example 1 below extend those of Applicant's previous work (see, Churchward-Venne T A, Burd N A, Mitchell C J, et al. Supplementation of a suboptimal protein dose with leucine or essential amino acids: effects on myofibrillar protein synthesis at rest and following resistance exercise in men. The Journal of physiology (2012)) by demonstrating that within the context of a mixed macronutrient beverage, a suboptimal protein dose (6.25 g) supplemented with a higher proportion of leucine (5.0 g total) was as effective at stimulating increased myofibrillar protein synthesis rates as a dose of protein (25 g) able to induce a maximal stimulation of myofibrillar protein synthesis rates following resistance exercise (see, Moore D R, Robinson M J, Fry J L, et al. Ingested protein dose response of muscle and albumin protein synthesis after resistance exercise in young men. The American journal of clinical nutrition 2009; 89(1):161-8).

As is disclosed in Example 1 below, the protein and amino acids were co-ingested with carbohydrates (“CHO”) and fat. While all treatments were energy-matched, the positive control (W25) contained more whey protein (25 g) and less CHO than the other treatments, which were isonitrogenous and macronutrient matched (see, Table 2). In Applicant's previous studies (see, Churchward-Venne T A, Burd N A, Mitchell C J, et al. Supplementation of a suboptimal protein dose with leucine or essential amino acids: effects on myofibrillar protein synthesis at rest and following resistance exercise in men. The Journal of physiology 2012) in which protein and free amino acids were ingested in isolation, supplementing 6.25 g of whey to contain 3.0 g of leucine induced peak blood amino acid concentrations of ˜550.0 μM, whereas in this study, the same protein dose supplemented up to 5.0 g leucine was necessary to achieve similar peak blood leucine concentrations when co-ingested with CHO and fat as part of a mixed macronutrient beverage. Thus, as has been reported previously (see, Burke L M, Winter J A, Cameron-Smith D, Enslen M, Farnfield M, Decombaz J. Effect of intake of different dietary protein sources on plasma amino Acid profiles at rest and after exercise. International journal of sport nutrition and exercise metabolism 2012; 22(6):452-62; and Staples A W, Burd N A, West D W, et al. Carbohydrate does not augment exercise-induced protein accretion versus protein alone. Medicine and science in sports and exercise 2011; 43(7):1154-61), co-ingestion of protein with additional macronutrients attenuates the postprandial rise in blood amino acid concentrations. On the other hand, W6+High-Leu and W25 were associated with a more prolonged increase in blood leucine and ΣEAA respectively compared with the other treatments (see, FIG. 3), which Applicant speculates may have served as a nutrient signal to maintain the increased myofibrillar protein synthesis rates observed over the late 1.5-4.5 hours post-exercise period.

While several studies have assessed the effects of protein-CHO co-ingestion on muscle protein synthesis rates (see, Staples A W, Burd N A, West D W, et al. Carbohydrate does not augment exercise-induced protein accretion versus protein alone. Medicine and science in sports and exercise 2011; 43(7):1154-61; and Koopman R, Beelen M, Stellingwerff T, et al. Coingestion of carbohydrate with protein does not further augment postexercise muscle protein synthesis. American journal of physiology Endocrinology and metabolism 2007; 293(3):E833-42), few studies have examined the muscle protein synthesis response following physiological (i.e., a single bolus) co-ingestion of protein, CHO, and fat (see, Elliot T A, Cree M G, Sanford A P, Wolfe R R, Tipton K D. Milk ingestion stimulates net muscle protein synthesis following resistance exercise. Medicine and science in sports and exercise 2006; 38(4):667-74). Although the addition of CHO to protein does not further stimulate increased muscle protein synthesis rates when ample protein is provided (see, Staples A W, Burd N A, West D W, et al. Carbohydrate does not augment exercise-induced protein accretion versus protein alone. Medicine and science in sports and exercise 2011; 43(7):1154-61; and Koopman R, Beelen M, Stellingwerff T, et al. Coingestion of carbohydrate with protein does not further augment postexercise muscle protein synthesis. American journal of physiology Endocrinology and metabolism 2007; 293(3):E833-42), it is not clear whether insulin can enhance muscle protein synthesis rates following intake of a suboptimal protein dose in the young. In Example 1 disclosed below, Applicant observed robust increases in myofibrillar protein synthesis rates following ingestion of W6, consisting of only 6.25 g whey protein, but co-ingested with 35.0 g CHO. Whether the myofibrillar protein synthesis response to W6 was enhanced by addition of CHO or whether only a small amount of leucine (0.75 g) serves as a sufficient nutrient signal to stimulate increased myofibrillar protein synthesis rates in young healthy individuals would require further investigation. The impact of protein-fat co-ingestion on myofibrillar protein synthesis rates compared to isolated protein feeding also warrants further investigation.

In conclusion, the results set forth in Example 1 below demonstrate that when a ‘suboptimal’ dose of protein (6.25 g), is supplemented leucine (W6+High-Leu) rates of myofibrillar protein synthesis equivalent in both magnitude and duration to those observed following ingestion of an energy-matched beverage containing saturating, for myofibrillar protein synthesis, dose of 25 g of whey protein (see, Moore D R, Robinson M J, Fry J L, et al. Ingested protein dose response of muscle and albumin protein synthesis after resistance exercise in young men. The American journal of clinical nutrition 2009; 89(1):161-8). These findings demonstrate that within the context of mixed macronutrient intake, suboptimal protein doses can be made more effective in stimulating myofibrillar protein synthesis through the addition of a high proportion of free leucine. This may be of importance in the development of nutritional formulations designed to promote skeletal muscle anabolism which may be of particular significance to individuals in whom total protein intake is restricted or inadequate.

The skilled artisan will appreciate that the enhanced myofibrillar protein synthesis may result upon administration of the present compositions to any number of different consumers including, for example, the very young, young adults, elderly adults, individuals undergoing physical rehabilitation, athletes, convalescents, etc. In an embodiment, the compositions may be administered immediately following a resistance exercise.

In an embodiment, the nutritional compositions of the present claims may be mixed macronutrient compositions (i.e., including a source of fats and a source of carbohydrates) and may include (i) an amount of whey protein that is not capable of enhancing myofibrillar protein synthesis when ingested by itself and (ii) free leucine. The composition may include at least about 5.0 g total leucine per dose of the compositions. In this regard, the composition may include, for examples, at least about 4.25 g added leucine per dose and about 6.25 g whey protein per dose (0.75 g leucine).

Physiological inference from increased skeletal muscle protein fractional synthesis rates (“FSR”) (see, Howarth K R, Moreau N A, Phillips S M & Gibala M J. (2009). Co-ingestion of protein with carbohydrate during recovery from endurance exercise stimulates skeletal muscle protein synthesis in humans. J Appl Physiol 106, 1036-1037; and Breen L, Philp A, Witard O C, Jackman S R, Selby A, Smith K, Baar K & Tipton K D. (2011). The influence of carbohydrate-protein co-ingestion following endurance exercise on myofibrillar and mitochondrial protein synthesis. J Physiol 589, 4011-4025) and regulation of myofibrillar and ECM developmental genes (see, Rowlands D S, Thomson J S, Timmons B W, Raymond F, Fuerholz A, Mansourian R, Zawhlen M C, Metairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020) in response to milk protein ingestion following exercise suggests these molecular responses may provide the basis for an improvement in subsequent performance demonstrated previously in rats and humans. See, D'Antona G, Ragni M, Cardile A, Tedesco L, Dossena M, Bruttini F, Caliaro F, Corsetti G, Bottinelli R, Carruba M O, Valerio A & Nisoli E. (2010). Branched-chain amino acid supplementation promotes survival and supports cardiac and skeletal muscle mitochondrial biogenesis in middle-aged mice. Cell Metabol 12, 362-372; and Thomson J S, Ali A & Rowlands D S. (2011). Leucine-protein supplemented recovery feeding enhances subsequent cycling performance. Appl Physiol Nutr Metabol 36, 242-253). Despite some evidence, understanding of the molecular processes responding to protein or amino acid ingestion that facilitate skeletal muscle recovery from and adaptation to intense endurance exercise is still at a relative infancy.

Following resistance exercise, the ingestion of ˜20-25 g of egg protein was sufficient to saturate mixed-muscle FSR. See, Moore D R, Tang J E, Burd N A, Rerecich T, Tarnopolsky M A & Phillips S M. (2009b). Differential stimulation of myofibrillar and sarcoplasmic protein synthesis with protein ingestion at rest and after resistance exercise. J Physiol 587, 897-904. In contrast, the ingested protein dose required to saturate muscle protein synthesis during recovery from endurance exercise is unknown. Leucine is the most potent trigger for increased skeletal muscle FSR, in part, by strongly stimulating translation initiation through mTORC1. See, Anthony J C, Yoshizawa F, Anthony T G, Vary T C, Jefferson L S & Kimball S R. (2000). Leucine stimulates translation initiation in skeletal muscle of postabsorptive rats via a rapamycin-sensitive pathway. J Nutr 130, 2413-2419. Leucine may also benefit skeletal muscle growth via attenuated proteolysis (see, Lunn W R, Pasiakos S M, Colletto M R, Karfonta K E, Carbone J W, Anderson J M & Rodriguez N R. (2012). Chocolate milk and endurance exercise recovery: protein balance, glycogen, and performance. Med Sci Sports Exerc 44, 682-691) and lower the total ingested nitrogen load required to increase FSR and protein balance (see, Churchward-Venne T A, Burd N A, Mitchell C J, West D W D, Philp A, Marcotte G R, Baker S K, Baar K & Phillips S M. (2012). Supplementation of a suboptimal protein dose with leucine or essential amino acids: effects on myofibrillar protein synthesis at rest and following resistance exercise in men. J Physiol 590, 2751-2765). Indeed, the addition of 3 g of leucine to low whey protein (6.25 g) ingested after resistance exercise led to similar 1-3 hour post-exercise myofibrillar FSR, compared with 25 g of whey protein. See, Id. Therefore, the first objective of the study of Example 2 was to determine the effect of varying the protein-leucine dose co-ingested with carbohydrate on the rate of myofibrillar FSR and on mTORC1 signaling following prolonged strenuous endurance exercise.

Recent nutritional transcriptomics data has revealed that post-exercise protein ingestion regulates skeletal muscle gene expression modulating myofibril remodeling (e.g., cytoskeleton and ECM), complement-mediated immunity and macrophage activation, and myocellular energy metabolism. See, Rowlands D S, Thomson J S, Timmons B W, Raymond F, Fuerholz A, Mansourian R, Zawhlen M C, Métairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020. With respect to energy metabolism, recent animal data also showed increased mitochondrial respiratory capacity and endurance performance associated with chronic branched-chain amino-acid supplementation. See, D'Antona G, Ragni M, Cardile A, Tedesco L, Dossena M, Bruttini F, Caliaro F, Corsetti G, Bottinelli R, Carruba M O, Valerio A & Nisoli E. (2010). Branched-chain amino acid supplementation promotes survival and supports cardiac and skeletal muscle mitochondrial biogenesis in middle-aged mice. Cell Metabol 12, 362-372. Evidence for nutrition-modulated inflammatory leukocyte activity is interesting because macrophages are crucial for phagocytosis of damaged tissue and stimulation of successful regeneration and myogenesis after exercise. See, Arnold L, Henry A, Poron F, Baba-Amer Y, van Rooijen N, Plonquet A, Gherardi R K & Chazaud B. (2007). Inflammatory monocytes recruited after skeletal muscle injury switch into anti-inflammatory macrophages to support myogenesis. J Exp Med 204, 1057-1069; and Tidball J G & Villalta S A. (2010). Regulatory interactions between muscle and the immune system during muscle regeneration. Am J Physiol Reg Int Comp Physiol 298, R1173-R1187. Therefore, the second objective of the study of Example 2 was to determine the primary protein-leucine dose-responsive molecular mechanisms guiding skeletal muscle recovery and adaptation following endurance exercise via interrogation of the transcriptome.

To assess the impact of the quantity of ingested whey protein with leucine on early phase molecular responses during recovery, Applicant associated plasma amino-acid concentrations and translational signaling activity with myofibrillar FSR, and determined the top-ranked dose regulated molecular systems via transcriptome interrogation. Applicant now reports new information on the dose of protein and leucine sufficient to near saturate myofibrillar FSR. In addition, Applicant extends the synthesis data to provide novel in vivo molecular insight into the effect of protein-leucine feeding on the early-phase adaptive regeneration response to an acute bout of intense endurance exercise.

As is shown in Example 2, in a crossover study design, twelve trained men completed 100 minutes of high-intensity cycling, then ingested 70/15/180/30 g protein/leucine/carbohydrate/fat (“15LEU”), 23/5/180/30 g (“5LEU”) or 0/0/274/30 g (“CON”) beverages in four (4) servings during the first 90 minutes of a 240-minute monitored recovery period. FSR was near-maximally stimulated with 5LEU (33% vs CON 90% confidence limits±12%), as tripling ingested protein-leucine dose (15LEU) only negligibly increased FSR (13%±12% vs 5LEU) despite augmented mTOR-p70S6K-rpS6 pathway activity. The top functional modular network in the 15LEU-5LEU dose contrast at 30 minutes was pro-inflammatory, centered on interleukin (I L) 1β, and programmed increased leukocyte migration and differentiation. The 5LEU and 15LEU vs CON feedings promoted cytostasis and increased cell viability with a myogenic signature. By 240 minutes, a protein-leucine dose sensitive IL6-centred anti-inflammatory and promyogenic transcriptome (associated with NF-Kappa-β and SMAD pathway activity inhibition) guided decreased leukocyte migration, and increased apoptosis of immune and muscle cells, and cell metabolism, suggesting progression through early-phase regeneration. Ingesting 24 g of protein and 5 g leucine nearly saturated post-exercise myofibrillar FSR, and simulated an early inflammatory transcriptome common to skeletal muscle regeneration biology that was accentuated with a 3-fold higher protein-leucine dose.

Irrespective of quantity, the co-ingestion of protein and leucine with carbohydrate and fat in the first 90-minutes following strenuous endurance exercise substantially increased (33-51%) myofibrillar FSR compared with carbohydrate and fat alone. The amino acid content of the 5LEU beverage represented the a priori estimated minimum quantity thought likely to induce near maximal myofibrillar FSR (see, Moore D R, Robinson M J, Fry J L, Tang J E, Glover E I, Wilkinson S B, Prior T, Tarnopolsky M A & Phillips S M. (2009a). Ingested protein dose response of muscle and albumin protein synthesis after resistance exercise in young men. Am J Clin Nutr 89, 161-168), whereas the 15LEU beverage was equivalent to that seen recently to enhance subsequent performance 36-hours later (see, Thomson J S, Ali A & Rowlands D S. (2011). Leucine-protein supplemented recovery feeding enhances subsequent cycling performance. Appl Physiol Nutr Metabol 36, 242-253). Indeed, tripling the protein-leucine dose caused a likely bioequivalent increase in mean FSR (only 13% greater). However, bioinformatic interrogation of the microarrays revealed a pro-inflammatory transcriptome associated with increased leukocyte migration most evident with the 15LEU at 30 minutes into recovery, reverting by 240 minutes to an anti-inflammatory promyogenic molecular programme with both protein-leucine quantities, relative to control. The transcriptome suggests protein-leucine feeding upregulated an early-phase myeloid-cell associated regeneration response reflecting wound-healing biology. See, Tidball J G & Villalta S A. (2010). Regulatory interactions between muscle and the immune system during muscle regeneration. Am J Physiol Reg Int Comp Physiol 298, R1173-R1187. Associated earlier restorative remodeling of skeletal muscle extracellular matrix and membrane stability may explain attenuated concentrations of muscle-membrane damage marker in the blood (creatine kinase) previously observed in the days following post-exercise protein-leucine feeding. See, Thomson J S, Ali A & Rowlands D S. (2011). Leucine-protein supplemented recovery feeding enhances subsequent cycling performance. Appl Physiol Nutr Metabol 36, 242-253; and Nelson A R, Phillips S M, Stellingwerff T, Rezzi S, Bruce S J, Breton I, Thorimbert A, Guy P A, Clarke J, Broadbent S & Rowlands D S. (2012). A protein-leucine supplement increases BCAA and nitrogen turnover but not performance Med Sci Sports Exerc 44, 57-68. Combined with increased protein synthesis and a myogenic transcriptome, these mechanisms could, in part, explain the reported improvement in performance following 3-days of ingesting similar quantities of protein and leucine after intense endurance cycling. See, Thomson J S, Ali A & Rowlands D S. (2011). Leucine-protein supplemented recovery feeding enhances subsequent cycling performance. Appl Physiol Nutr Metabol 36, 242-253.

As shown by Example 2, myofibrillar FSR in the 5LEU condition was bioequivalent to the 15 LEU FSR, despite 3-fold greater amino-acid provision. Similarly, ingesting 20 g of egg protein following resistance exercise stimulated ˜95% of the mean mixed-muscle FSR observed with 40 g feeding. See, Moore D R, Robinson M J, Fry J L, Tang J E, Glover E I, Wilkinson S B, Prior T, Tarnopolsky M A & Phillips S M. (2009a). Ingested protein dose response of muscle and albumin protein synthesis after resistance exercise in young men. Am J Clin Nutr 89, 161-168. Furthermore, following resistance exercise in men, 4×20 g feedings ingested 3 hours apart led to higher myofibrillar FSR than 8×10 g every 90 minutes, or 2×40 g every 6 hours. See, Areta J L, Burke L M, Ross M L, Camera D M, West D W D, Broad E M, Jeacocke N A, Moore D R, Stellingwerff T, Phillips S M, Hawley J & Coffey V G. (2013). Timing and distribution of protein ingestion during prolonged recovery from resistance exercise alters myofibrillar protein synthesis. The Journal of Physiology. Similar experiments are required to determine the ingested dietary protein requirement to near maximally stimulate skeletal muscle myofibril protein synthesis in lean healthy men following endurance exercise.

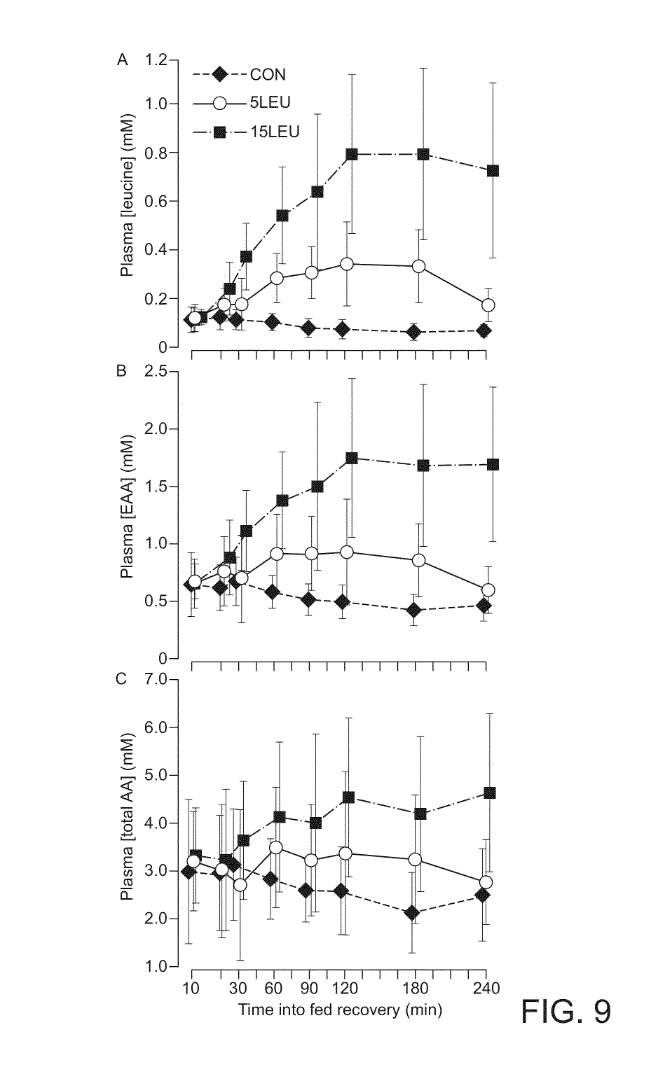

In discordance with the FSR outcome, mTOR-pathway activity was stimulated to the greatest extent in the 15LEU vs 5LEU condition, with peak phosphorylation of mTOR and p70S6K at 30 minutes, and 4E-BP1 and rpS6 at 240 min. Delayed phosphorylation of 4E-BP1 and rpS6 until 240-minutes in the 5LEU suggests translation initiation signaling may have been retarded until more nutrition was ingested; the delayed increase in plasma leucine concentration in 5LEU vs. 15LEU also supports this contention. See, FIG. 9. Therefore, the positive dose-association with phosphorylated p70S6K at 30 minutes and rpS6 at 240 minutes, and the positive correlations between plasma leucine concentration and p70S6K, rpS6, and 4E-BP1γ most likely resulted from the ingested dose-related rise in plasma leucine. See, Crozier S J, Kimball S R, Emmert S W, Anthony J C & Jefferson L S. (2005). Oral leucine administration stimulates protein synthesis in rat skeletal muscle. J Nutr 135, 376-382). Atherton et al. also reported discordance between mTORC1 signaling and myofibrillar FSR at rest following ingestion of 48 g of whey protein. See, Atherton P J, Etheridge T, Watt P W, Wilkinson D, Selby A, Rankin D, Smith K & Rennie M J. (2010). Muscle full effect after oral protein: time-dependent concordance and discordance between human muscle protein synthesis and mTORC1 signaling. Am J Clin Nutr 92, 1080-1088. In their work, myofibrillar FSR peaked 46-90 minutes post-feeding and coinciding with p70S6K and 4EBP1 phosphorylation before rapidly returning to baseline despite the continued availability of plasma and muscle leucine and EAAs, and preceding the dephosphorylation of p70S6K and 4EBP1. See, Id. In agreement with Atherton et al., the study of Example 2 shows that EAA signaling potency for FSR appears limited to regulation of mTOR-S6K-rpS6 phosphorylation (translation initiation) rather than elongation via eEF2. Therefore, mTOR pathway signaling associations correlate with, but do not quantitatively predict, myofibrillar FSR outcomes in response to protein feeding after exercise. Nevertheless, the dose-responsive mTORC1-pathway activity may regulate other cellular outcomes through its function as a highly conserved regulator of response to nutrients and growth; e.g., regulation of polymerase I expression, cell cycle activity, metabolism and cell growth. See, Mayer C, Zhao J, Yuan X & Grummt I (2004). mTOR-dependent activation of the transcription factor TIF-IA links rRNA synthesis to nutrient availability. Genes Devel 18, 423-434; and Nader G A, McLoughlin T J & Esser K A. (2005). mTOR function in skeletal muscle hypertrophy: increased ribosomal RNA via cell cycle regulators. Am J Physiol Cell Physiol 289, C1457-C1465; and Duvel K, Yecies J L, Menon S, Raman P, Lipovsky A I, Souza A L, Triantafellow E, Ma Q, Gorski R, Cleaver S, Vander Heiden M G, MacKeigan J P, Finan P M, Clish C B, Murphy L O & Manning B D. (2010). Activation of a metabolic gene regulatory network downstream of mTOR complex 1. Mol Cell 39, 171-183.

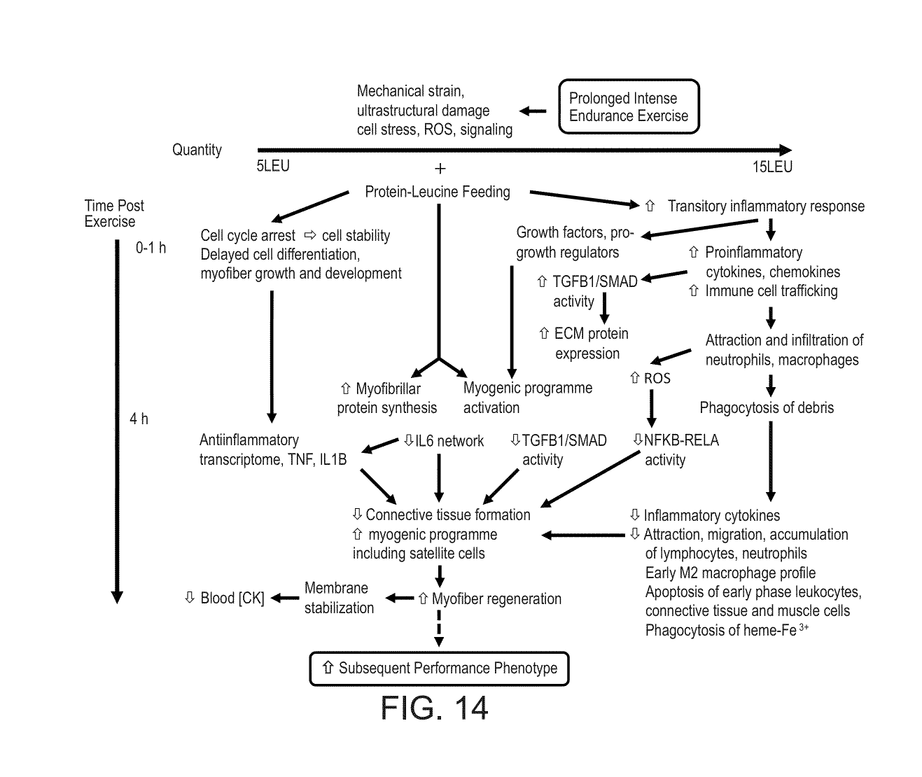

As shown by Example 2, the 15LEU-5LEU dose feeding contrast produced a pro-inflammatory transcriptome at 30 minutes with IL1β-centered connectivity to increased leukocyte invasion and cell differentiation molecular functions, while both protein-leucine treatments induced expression congruent with increased cell stability. By 240 minutes in both 15LEU and 5LEU conditions, attenuation of the initial pro-inflammatory impulse, decreased leukocyte migration coupled with increased myeloid and muscle cell apoptosis and formation of promyogenic networks, suggested progression through the skeletal muscle regeneration response. The classical inflammatory response of skeletal muscle tissue to unusual loading or injury involves the release and increased tissue concentration of growth factors and cytokines, stimulation of resident and infiltrating leukocytes, fibroblast production of ECM components that are degraded as regeneration proceeds (see, Pizza F X, Peterson J M, Baas J H & Koh T J. (2005). Neutrophils contribute to muscle injury and impair its resolution after lengthening contractions in mice. J Physiol 562, 899-913; and Burks T N & Cohn R D. (2011). Role of TGF-beta signaling in inherited and acquired myopathies. Skel Muscle 1, 19), and the activation, proliferation, and differentiation of satellite cells to either fuse with existing fibers or with other myogenic cells to generate new fibers (see, Tidball J G & Villalta S A. (2010). Regulatory interactions between muscle and the immune system during muscle regeneration. Am J Physiol Reg Int Comp Physiol 298, R1173-R1187). This early response initiates cleanup of cellular debris within the muscle, contributing to membrane and oxidative damage, and leads to the release of cytokines to promote myogenesis, inflammatory resolution, and regeneration. See, Burks T N & Cohn R D. (2011). Role of TGF-beta signaling in inherited and acquired myopathies. Skel Muscle 1, 19. Here, Applicant presents new evidence that myeloid-cell associated skeletal muscle regeneration processes were activated by postexercise protein-leucine feeding, and that this effect is dose modulated. See, FIG. 14.

Responding to pro-inflammatory signaling, M1 macrophages (and neutrophils) can increase muscle damage via reactive oxygen species (ROS) including nitric oxide (see, Villalta S A, Nguyen H X, Deng B, Gotoh T & Tidball J G. (2009). Shifts in macrophage phenotypes and macrophage competition for arginine metabolism affect the severity of muscle pathology in muscular dystrophy. Human Mol Gen 18, 482-496); evidence for increased ROS was resolved from the 15LEU-5LEU transcriptome. Increased nitric oxide may also promote greater leukocyte adhesion (see, Kubes P, Suzuki M & Granger D N. (1991). Nitric oxide: an endogenous modulator of leukocyte adhesion. Proc Nat Acad Sci 88, 4651-4655), another step in the classical muscle regeneration response (see, Tidball J G & Villalta S A. (2010). Regulatory interactions between muscle and the immune system during muscle regeneration. Am J Physiol Reg Int Comp Physiol 298, R1173-R1187). The 15LEU-5LEU transcriptome also increased CD14 expression, a toll receptor complex component, and activation of toll receptor 9 was predicted from upstream regulatory analysis, complementing current transcriptome evidence for activation of NFKB, cytokine secretion (C C and CXC chemokines) and the inflammatory response. See, Boyd J H, Divangahi M, Yahiaoui L, Gvozdic D, Qureshi S & Petrof B J. (2006). Toll-like receptors differentially regulate C C and CXC chemokines in skeletal muscle via NF-kappaB and calcineurin. Infect Imm 74, 6829-6838). Additionally, increased S100A10 and CD163 expression at 30 minutes suggests greater macrophage migration and M2 macrophage activity. See, O'Connell P A, Surette A P, Liwski R S, Svenningsson P & Waisman D M. (2010). S100A10 regulates plasminogen-dependent macrophage invasion. Blood 116, 1136-1146. Internalization and breakdown of the ligated haemoglobin-CD163 or CD163-haptaglobin complexes can contribute toward lowering extracellular haemoglobin and associated free radical production and cellular damage, while also promoting anti-inflammatory cytokine release. See, Moestrup S K & Moller H J. (2004). CD163: a regulated hemoglobin scavenger receptor with a role in the anti-inflammatory response. Ann Med 36, 347-354. Unfortunately, muscle sample damage by freeze artefact prevented subsequent immunohistochemical quantification of macrophage and neutrophil antigen content within the skeletal muscle. Nevertheless, future validation work should also determine the physiological processes regulated by amino acids directing acute phase neutrophil and macrophage activation including pro-inflammatory cytokine production and stimulation of phagocytosis involved in the early regeneration response. For example, increased CD68 expression with 15LEU feeding could suggest macrophage activation. CD68 is a functionally important glycoprotein involved in phagocytosis and cytokine release. See, Zouaoui Boudjeltia K, Moguilevsky N, Legssyer I, Babar S, Guillaume M, Delree P, Vanhaeverbeek M, Brohee D, Ducobu J & Remade C. (2004). Oxidation of low density lipoproteins by myeloperoxidase at the surface of endothelial cells: an additional mechanism to subendothelium oxidation. Biochem Biophys Res Com 325, 434-438. Increased neutrophil invasion and myeloperoxidase release may have induced cell membrane damage and oxidation of low-density lipoproteins. See, Id. The binding of modified LDL to CD68 activates phagocytosis and muscle cell lysis by M1 macrophages. See, Nguyen H X & Tidball J G. (2003). Interactions between neutrophils and macrophages promote macrophage killing of rat muscle cells in vitro. J Physiol 547, 125-132. Coupled with increased cytokine production, the interaction of neutrophils and macrophages regulated by feeding maybe one of several important regulatory processes in muscle recovery from intense exercise. See, Pizza F X, Koh T J, McGregor S J & Brooks S V. (2002). Muscle inflammatory cells after passive stretches, isometric contractions, and lengthening contractions. J Appl Physiol 92, 1873-1878; and Pizza F X, Peterson J M, Baas J H & Koh T J. (2005). Neutrophils contribute to muscle injury and impair its resolution after lengthening contractions in mice. J Physiol 562, 899-913; and Arnold L, Henry A, Poron F, Baba-Amer Y, van Rooijen N, Plonquet A, Gherardi R K & Chazaud B. (2007). Inflammatory monocytes recruited after skeletal muscle injury switch into anti-inflammatory macrophages to support myogenesis. J Exp Med 204, 1057-1069.

A molecular programme associated with phagocyte invasion prior to activation of targeted cell destruction and debris removal was evident from the biphasic transcriptome observed in the CDKN1A (p21), MYC, and GADD45A network hubs responding to both protein-leucine doses; this could suggest increased then decreased cellular stability and viability through cell cycle arrest and cell apoptosis. See, Burks T N & Cohn R D. (2011). Role of TGF-beta signaling in inherited and acquired myopathies. Skel Muscle 1, 19. Apoptotic leukocytes stimulate clearance of cellular debris by the macrophages, while concomitantly silencing the pro-inflammatory/phagocytoxic action of the macrophages. See, Ariel A & Serhan C N. (2012). New lives given by cell death: Macrophage differentiation following their encounter with apoptotic leukocytes during the resolution of inflammation. Frontiers Immunol 3, 1-6. GADD45A responds to cell stress and is an important regulator of anabolic signaling and energy homeostasis. See, Ebert S M, Dyle M C, Kunkel S D, Bullard S A, Bongers K S, Fox D K, Dierdorff J M, Foster E D & Adams C M. (2012). Stress-induced skeletal muscle Gadd45a expression reprograms myonuclei and causes muscle atrophy. J Biol Chem 287, 27290-27301. P21 induces cell cycle arrest. See, Lokireddy S, McFarlane C, Ge X, Zhang H, Sze S K, Sharma M & Kambadur R. (2011). Myostatin induces degradation of sarcomeric proteins through a Smad3 signaling mechanism during skeletal muscle wasting. Mol Endocrinol 25, 1936-1949. MYC has a widespread impact on the transcriptome including regulation of cell growth arrest and adhesion, metabolism, ribosome biogenesis, protein synthesis, and mitochondrial function. See, Dang C V, O'Donnell K A, Zeller K I, Nguyen T, Osthus R C & Li F. (2006). The c-Myc target gene network. Sem Cancer Biol 16, 253-264. Therefore, the data suggest that one of the early responses to post-exercise protein feeding in trained skeletal muscle is cell stabilization, which may assist in maintenance of homeostasis and protein synthetic machinery prior to activation of the restorative programme.

As shown in Example 2, and at 240 minutes, both 15LEU and 5LEU doses mediated an IL6-centred anti-inflammatory transcriptome consistent with M1 to M2 macrophage phenotype shift (see, Tidball J G & Villalta S A. (2010). Regulatory interactions between muscle and the immune system during muscle regeneration. Am J Physiol Reg Int Comp Physiol 298, R1173-R1187) and coupling to a promyogenic response involving satellite cells (see, Serrano A L, Baeza-Raja B, Perdiguero E, Jardí M & Muñoz-Cánoves P. (2008). Interleukin-6 Is an essential regulator of satellite cell-mediated skeletal muscle hypertrophy. Cell Metabol 7, 33-44). The transcriptome associated with inhibition of NFKB signaling by protein-leucine may reduce migration of the p65 (RELA)-p50 (NFKB1) dimers to the nucleus to regulate cyclinD1; effects causing increased differentiation and reduced pro-inflammatory cytokine expression (IL6, TNFα, IL1). See, Bakkar N & Guttridge D C. (2010). NF-kappaB signaling: a tale of two pathways in skeletal myogenesis. Physiol Rev 90, 495-511. Moreover, current increased MYOD1 expression and decreased TGFβ-SMAD associated transcriptome activity (15LEU only) also suggests myogenic differentiation: MYOD stimulates cell cycle re-entry by inducing NFKB-complex relocalization from the nucleus to the cytoplasm (see, Parker M H, von Maltzahn J, Bakkar N, Al-Joubori B, Ishibashi J, Guttridge D & Rudnicki M A. (2012). MyoD-dependent regulation of NF-κB activity couples cell-cycle withdrawal to myogenic differentiation. Skel Muscle 2, 6) and SMAD pathway inhibition is antifibrotic and promyogenic (see, Burks T N & Cohn R D. (2011). Role of TGF-beta signaling in inherited and acquired myopathies. Skel Muscle 1, 19). Rowlands et al. also reported increased expression of myogenic differentiation factors (MYOD1, MEF5, MYOG) in skeletal muscle with protein ingestion following intense cycling in trained men. See, Rowlands D S, Thomson J S, Timmons B W, Raymond F, Fuerholz A, Mansourian R, Zawhlen M C, Métairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020. In murine skeletal muscle, increased inflammatory cell content and satellite cell activation displaying strong staining for MYOD preceded extensive myofibrillar regeneration in response to injury. See, Yan Z, Choi S, Liu X, Zhang M, Schageman J J, Lee S Y, Hart R, Lin L, Thurmond F A & Williams R S. (2003). Highly coordinated gene regulation in mouse skeletal muscle regeneration. J Biol Chem 278, 8826-8836.

The magnitude of ECM gene expression reflects the degree of functional remodeling in response to endurance training. See, Timmons J, Jansson E, Fischer H, Gustafsson T, Greenhaff P, Ridden J, Rachman J & Sundberg C. (2005). Modulation of extracellular matrix genes reflects the magnitude of physiological adaptation to aerobic exercise training in humans. BMC Biol 3, 19. Therefore, noteworthy was the substantial biphasic ECM gene expression and the TGFβ-SMAD signaling pathway transcriptome activated at 30 minutes with 15LEU, then relatively inhibited by 240 minutes vs CON. SMAD pathway activation and extensive up-regulation of ECM component expression would normally contribute towards scar formation (see, Li Y, Foster W, Deasy B M, Chan Y, Prisk V, Tang Y, Cummins J & Huard J. (2004). Transforming growth factor-β1 induces the differentiation of myogenic cells into fibrotic cells in injured skeletal muscle: A key event in muscle fibrogenesis. Am J Pathol 164, 1007-1019) negatively affecting skeletal muscle regeneration by inhibiting satellite cell proliferation and myofiber fusion (see, Allen R E & Boxhorn L K. (1987). Inhibition of skeletal muscle satellite cell differentiation by transforming growth factor-beta. J Cell Physiol 133, 567-572). However, inhibition of SMAD pathway activated transcriptome by 240 minutes in the 15LEU condition suggests any profibrotic response was more likely transient and guiding an impulse of ECM protein deposition (e.g. collagen, proteoglycans, laminin) to support basement membrane and other ECM remodeling. See, Chen X & Li Y. (2009). Role of matrix metalloproteinases in skeletal muscle: Migration, differentiation, regeneration and fibrosis. Cell Adhesion Migration 3, 337-341. Accordingly, the accommodating biphasic expression of CTGF and other fibrotic regulators, e.g., the proteoglycan DCN which antagonizes TBFβ and IGF1, may have invoked accelerated muscle regeneration by modulating inflammatory cytokines and chemokines. See, Pelosi L, Giacinti C, Nardis C, Borsellino G, Rizzuto E, Nicoletti C, Wannenes F, Battistini L, Rosenthal N, Molinaro M & Musaro A. (2007). Local expression of IGF-1 accelerates muscle regeneration by rapidly modulating inflammatory cytokines and chemokines. FASEB J21, 1393-1402; and Brandan E, Cabello-Verrugio C & Vial C. (2008). Novel regulatory mechanisms for the proteoglycans decorin and biglycan during muscle formation and muscular dystrophy. Matrix Biol 27, 700-708. Other recent evidence also supports a role for post-exercise protein feeding in ECM turnover via regulated expression of matrix metallopeptidases and inhibitors (increased MMP9, MMP13, MMP19; decreased TIMP1, TIMP2) (see, Rowlands D S, Thomson J S, Timmons B W, Raymond F, Fuerholz A, Mansourian R, Zawhlen M C, Métairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020), which control basement membrane degradation facilitating the recruitment of myogenic, myeloid, vascular, and fibroblastic cells to damaged muscle (see, Mann C, Perdiguero E, Kharraz Y, Aguilar S, Pessina P, Serrano A L & Munoz-Canoves P. (2011). Aberrant repair and fibrosis development in skeletal muscle. Skel Muscle 1, 21), ECM remodeling, and the interface for leukocytes, cytokines, growth factors (see, Chen X & Li Y. (2009). Role of matrix metalloproteinases in skeletal muscle: Migration, differentiation, regeneration and fibrosis. Cell Adhesion Migration 3, 337-341). Because it was not possible to decipher the cell types from which the nutrition-responsive transcriptome was most active, future work should consider in situ methods to quantify functional mRNA and protein expression in skeletal muscle cell types to define the cellular-specific role of amino acids in post-exercise regenerating skeletal muscle.

An inferential consideration is that the present nutrition responsive myeloid-cell associated transcriptome programme for tissue regeneration occurred after normal exercise in well-trained muscle. Passive stretches and isometric contractions elevated neutrophils without causing injury and offered protection from damage caused by subsequent lengthening contractions. See, Pizza F X, Koh T J, McGregor S J & Brooks S V. (2002). Muscle inflammatory cells after passive stretches, isometric contractions, and lengthening contractions. J Appl Physiol 92, 1873-1878. Therefore, intense concentric cycling exercise in trained muscle, while causing fatigue and free-radical associated damage is unlikely to have caused the substantial microstructural damage associated with heavy eccentric damage models used to study the inflammatory regeneration response (e.g. Paulsen G, Crameri R, Benestad H B, Fjeld J G, Morkrid L, Hallen J & Raastad T. (2010). Time course of leukocyte accumulation in human muscle after eccentric exercise. Med Sci Sports Exerc 42, 75-85). Furthermore, the skeletal muscle of athletes is constantly remodeling, which implies (a) the muscle is likely to exhibit a relatively modulated or moderated inflammatory and regeneration response, (b) is in a state of constant flux between damage and regeneration events, and (c) is integrating regeneration responses with the molecular programme necessary for increased homeostatic capacity required for improved contractile function. Because myeloid-associated regeneration processes responding to heavy exercise occur over several days to weeks (see, Pizza F X, Koh T J, McGregor S J & Brooks S V. (2002). Muscle inflammatory cells after passive stretches, isometric contractions, and lengthening contractions. J Appl Physiol 92, 1873-1878; and Paulsen G, Crameri R, Benestad H B, Fjeld J G, Morkrid L, Hallen J & Raastad T. (2010). Time course of leukocyte accumulation in human muscle after eccentric exercise. Med Sci Sports Exerc 42, 75-85; and Tidball J G & Villalta S A. (2010). Regulatory interactions between muscle and the immune system during muscle regeneration. Am J Physiol Reg Int Comp Physiol 298, R1173-R1187), and because there is evidence for an enhanced mitochondrial biogenic signature and adaptation over a longer time frame (see, D'Antona G, Ragni M, Cardile A, Tedesco L, Dossena M, Bruttini F, Caliaro F, Corsetti G, Bottinelli R, Carruba M O, Valerio A & Nisoli E. (2010). Branched-chain amino acid supplementation promotes survival and supports cardiac and skeletal muscle mitochondrial biogenesis in middle-aged mice. Cell Metabol 12, 362-372; and Rowlands D S, Thomson J S, Timmons B W, Raymond F, Fuerholz A, Mansourian R, Zawhlen M C, Metairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020). The collection of tissue samples for several days following a bout of strenuous exercise is required to gain a thorough appreciation of nutrition-mediated regeneration. Furthermore, possible accumulative effects of accelerated regeneration and metabolic adaptation with repeated post-training feeding warrant examination of the human skeletal-muscle performance phenotype responding to chronic feeding to provide practical validation to the molecular inference.

In conclusion, the study of Example 2 of the present disclosure shows that the ingestion of 23 g of whey protein and 5 g of leucine in the 90-minutes period following intense endurance exercise was sufficient to nearly saturate myofibrillar FSR, despite higher blood amino-acid availability and augmented muscle mTOR-p70S6K-rpS6 pathway activity with a 3-fold higher dose. The promyogenic regenerative programme inferred from interrogation of the transcriptome was apparent with both protein-leucine doses, but the higher quantity of nutrition primarily activated the pro-inflammatory transcriptome. As such, the dose of protein-leucine could be mechanistically instrumental in regulating the myeloid-associated regeneration processes, prospectively leading to better supercompensation of contractile function. Further research would be valuable to define the effects of post-exercise amino acid exposure and dose on function and role of myeloid cells, satellite cells and myofibers on the integrative of the complex molecular and cellular responses guiding adaptive regeneration in trained skeletal muscle following intense aerobic exercise.

The nutritional compositions of the present disclosure may be administered by any means suitable for human administration, and in particular for administration in any part of the gastrointestinal tract. Enteral administration, oral administration, and administration through a tube or catheter are all covered by the present disclosure. The nutritional compositions may also be administered by means selected from oral, rectal, sublingual, sublabial, buccal, topical, etc.

If the nutritional compositions are formulated to be administered orally, the compositions may be a liquid oral nutritional supplement (e.g., incomplete feeding) or a complete feeding. In this manner, the nutritional compositions may be administered in any known form including, for example, tablets, capsules, liquids, chewables, soft gels, sachets, powders, syrups, liquid suspensions, emulsions and solutions in convenient dosage forms. In soft capsules, the active ingredients are preferably dissolved or suspended in suitable liquids, such as fatty oils, paraffin oil or liquid polyethylene glycols. Optionally, stabilizers may be added. In an embodiment, the nutritional compositions are beverages.

The nutritional compositions of the present disclosure may be a source of either incomplete or complete nutrition. The nutritional compositions may also be used for short term or long term tube feeding.

Suitable nutritional composition formats according to the present disclosure include, for example, solutions, ready-for-consumption compositions (e.g. ready-to-drink compositions or instant drinks), liquid comestibles, soft drinks, juice, sports drinks, milk drinks, milk-shakes, yogurt drinks, soup, etc. In a further embodiment, the nutritional compositions may be manufactured and sold in the form of a concentrate, a powder, or granules (e.g. effervescent granules), which are diluted with water or other liquid, such as milk or fruit juice, to yield a ready-for-consumption composition (e.g. ready-to-drink compositions or instant drinks).

In addition to mixed macronutrients, the presently disclosed nutritional compositions may include a source of ω-3 and/or ω-6 fatty acids. Examples of sources of ω-3 fatty acids include, for example, fish oil, krill, plant sources of ω-3, flaxseed, walnut, and algae. Non-limiting examples of ω-3 fatty acids include α-linolenic acid (“ALA”), docosahexaenoic acid (“DHA”), and eicosapentaenoic acid (“EPA”). Non-limiting examples of ω-6 fatty acids include linoleic acid (“LA”), arachidonic acid (“ARA”).

In an embodiment, the nutritional compositions include a source of phytochemicals. Phytochemicals are non-nutritive compounds that are found in many fruits and vegetables, among other foods. There are thousands of phytochemicals that can be categorized generally into three main groups. The first group is flavonoids and allied phenolic and polyphenolic compounds. The second group is terpenoids, e.g., carotenoids and plant sterols. The third group is alkaloids and sulfur containing compounds. Phytochemicals are active in the body and, in general, act similarly to antioxidants. They also appear to play beneficial roles in inflammatory processes, clot formation, asthma, and diabetes. Researchers have theorized that to receive the most benefit from consumption of phytochemicals, they should be consumed as part of whole foods, because of the complex, natural combination and potentially synergistic effects. This may partially explain the health benefits associated with consumption of whole fruits and vegetables. Increased intake of fruits and vegetables is associated with reduced risk of many chronic diseases. In order to enhance the phytochemical profile of the present nutritional compositions, in an embodiment, the compositions include various fruits and vegetables containing these compounds.

In an embodiment, the nutritional compositions include a source of protein. The protein source may be dietary protein including, but not limited to animal protein (such as milk protein, meat protein or egg protein), vegetable protein (such as soy protein, wheat protein, rice protein, and pea protein), or combinations thereof. In an embodiment, the protein is selected from the group consisting of whey, chicken, corn, caseinate, wheat, flax, soy, carob, pea or combinations thereof. In another embodiment, the protein is pea protein or pea protein isolate.

The nutritional compositions of the present disclosure may also include a source of carbohydrates. Any suitable carbohydrate may be used in the present nutritional compositions including, but not limited to, sucrose, lactose, glucose, fructose, corn syrup solids, maltodextrin, modified starch, amylose starch, tapioca starch, corn starch or combinations thereof.

A source of fat may also be included in the present nutritional compositions. The source of fat may include any suitable fat or fat mixture. For example, the fat source may include, but is not limited to, vegetable fat (such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like), animal fats (such as milk fat), or combinations thereof. The source of fat may also be less refined versions of the fats listed above (e.g., olive oil for polyphenol content).

In an embodiment, the nutritional compositions further include one or more prebiotics. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

The nutritional compositions may further include one or more probiotics. Non-limiting examples of probiotics include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

One or more amino acids may also be present in the nutritional compositions. Non-limiting examples of amino acids include alanine, arginine, asparagine, aspartate, citrulline, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, or combinations thereof.

One or more antioxidants may also be present in the nutritional compositions. Non-limiting examples of antioxidants include astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, or combinations thereof.

The nutritional compositions may also include fiber or a blend of different types of fiber. The fiber blend may contain a mixture of soluble and insoluble fibers. Soluble fibers may include, for example, fructooligosaccharides, acacia gum, inulin, etc. Insoluble fibers may include, for example, pea outer fiber.

Methods of administering the nutritional compositions of the present disclosure are also provided. For example, in a general embodiment, a method for enhancing muscle protein synthesis in an individual in need of same is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In another embodiment, a method for enhancing muscle anabolism in an individual in need of same is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In yet another embodiment, a method for enhancing muscle protein synthesis in an athlete is provided. The method includes administering to the athlete a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In still yet another embodiment, a method for enhancing muscle protein synthesis in an elderly individual is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In another embodiment, a method for enhancing muscle protein synthesis in an individual undergoing muscle rehabilitation is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In yet another embodiment, a method for enhancing muscle protein synthesis in an individual undergoing physical therapy is provided. The method includes administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

In still yet another embodiment, a method for enhancing muscle protein synthesis in a young adult is provided. The method includes administering to the young adult a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose.

By way of example and not limitation, the following examples are illustrative of various embodiments of the present disclosure. The formulations and processes below are provided for exemplification only, and they can be modified by the skilled artisan to the necessary extent, depending on the special features that are desired.

### Example 1

### Subjects and Methods

Study Participants

Forty young adult males between 18-35 years of age were recruited to participate in a study conducted by Applicants. The characteristics of the study participants are presented in Table 1.

TABLE 1
Participant Characteristics

| Treatment Group | W6 + Low-Leu | W6 + BCAA | W6 | W25 | W6 + High-Leu |
|---|---|---|---|---|---|
| Age (yrs) | 20.4 (0.6) | 20.8 (0.8) | 20.5 (1.1) | 20.9 (0.6) | 19.5 (0.1) |
| Height (m) | 1.76 (0.02) | 1.83 (0.03) | 1.80 (0.03) | 1.76 (0.02) | 1.76 (0.03) |
| Weight (kg) | 77.7 (3.3) | 81.3 (3.8) | 79.4 (3.5) | 78.1 (2.8) | 79.4 (3.4) |
| BMI (kg/m2) | 25.0 (1.0) | 24.3 (0.8) | 24.5 (0.7) | 25.2 (1.0) | 25.7 (1.2) |
| Fat-free Mass (kg) | 64.2 (2.3) | 70.4 (2.9) | 66.6 (2.4) | 66.0 (2.8) | 64.8 (2.7) |
| Fat-mass (kg) | 13.6 (1.6) | 10.9 (1.2) | 12.9 (1.3) | 12.1 (0.8) | 14.6 (1.2) |
| 1-RM Strength (kg) | 71.3 (4.2) | 67.3 (3.6) | 64.5 (1.7) | 65.0 (3.2) | 59.4 (3.5) |

Values are mean ± SEM (n = 8 per treatment group)

In a double blind manner, participants were randomly assigned in a block design to one of five post-exercise nutritional treatment groups (described below) balanced for bodyweight. None of the study participants reported engaging in a structured program of resistance exercise within the last year, but reported being recreationally active about 2-3 times per week. Participants were deemed healthy based on responses to a routine health screening questionnaire. Each participant was informed of the purpose of the study, experimental procedures, and potential risks prior to providing written consent. The study was approved by the Hamilton Health Sciences Research Ethics Board and conformed to the standards for the use of human subjects in research as outlined in the most recent update of the Declaration of Helsinki. The study also conformed to standards established by the Canadian Tri-Council Policy on the ethical use of human subjects. See, Canadian Institutes of Health Research, Natural Sciences and Engineering Research Council of Canada, and Social Sciences and Humanities Research Council of Canada, Tri-Council Policy Statement: Ethical Conduct for Research Involving Humans. 2010.

Pretesting

Approximately one week prior to the experimental infusion trial, study participants underwent unilateral strength testing of the knee-extensor muscles. Participants performed a series of graded knee-extensions to determine their single repetition maximum strength (1-RM) with their self reported dominant leg using a seated knee-extension device. In addition, each participant underwent a whole-body dual-energy X-ray absorptiometry scan to measure body composition (see, Table 1). Participants were provided with pre-packaged standardized diets consumed two days immediately preceding the experimental infusion trial. Diets were designed to provide sufficient energy to maintain energy balance as determined by the Harris-Benedict equation and were adjusted using a moderate activity factor (1.4-1.6) to account for participants self-reported physical activity patterns. The macronutrient distribution of the diets was 55% carbohydrate, 30% fat, and 15% protein. Participants were instructed to consume all food and beverages provided and avoid consumption of food and beverages not provided as part of the standardized diet. Participants were instructed to abstain from strenuous physical exercise for 72 hours prior to the experimental infusion trial and to consume their evening meal no later than 20 00 hours prior to the trial.

Experiment

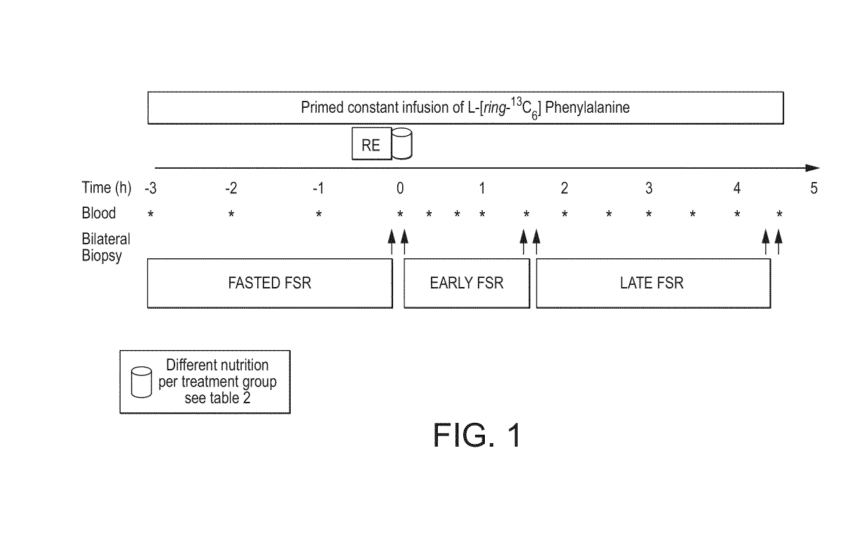

Participants reported to the lab at 0600 the morning of the experimental infusion trial following an overnight fast. A catheter was inserted into an antecubital vein and a baseline blood sample was taken before initiating a 0.9% saline drip to keep the catheter patent to allow for repeated arterialized blood sampling. Arterialized blood samples were obtained repeatedly during the infusion trial (see, FIG. 1) by wrapping a heating blanket around the forearm. See, Copeland K C, Kenney F A, Nair K S. Heated dorsal hand vein sampling for metabolic studies: a reappraisal. The American journal of physiology 1992; 263. Blood samples were collected into 4 ml heparinized evacuated tubes and chilled on ice. A second catheter was inserted into the antecubital vein of the opposite arm prior to initiating a primed continuous infusion (0.05 μmol·kg⁻¹·min⁻¹; 2.0 μmol·kg⁻¹ prime) of [ring-¹³C₆] phenylalanine. The infusate was passed through a 0.2-μm filter before entering the blood. The basal (“Fasted”) fractional synthetic rate (“FSR”) was calculated based on the ¹³C enrichment of mixed plasma proteins obtained from the pre-infusion blood sample and skeletal muscle biopsy following ˜3 hours of tracer incorporation. See, Tang J E, Moore D R, Kujbida G W, Tarnopolsky M A, Phillips S M. Ingestion of whey hydrolysate, casein, or soy protein isolate: effects on mixed muscle protein synthesis at rest and following resistance exercise in young men. J Appl Physiol 2009; 107(3):987-92; and Burd N A, West D W, Staples A W, et al. Low-load high volume resistance exercise stimulates muscle protein synthesis more than high-load low volume resistance exercise in young men. PloS one 2010; 5(8):e12033. Participants performed an acute bout of unilateral seated knee-extension resistance exercise consisting of 8 sets of 10-12 repetitions at ˜80% of their previously determined 1-RM with an inter-set rest-interval of 2 minutes.

Immediately following completion of the resistance exercise, participants underwent bilateral biopsies from both the rested and exercised leg and immediately ingested their designated nutrient treatment (see below). Bilateral biopsy samples were then obtained at 1.5 hours and 4.5 hours post-exercise from a rested fed (“FED”) and exercise-fed (“EX-FED”) leg. Muscle biopsies were obtained from the vastus lateralis muscle using a 5 mm Bergstrom needle custom adapted for manual suction under 2% xylocaine local anaesthesia. The tissue samples were freed from visible blood, fat, and connective tissue, and immediately frozen in liquid nitrogen for further analysis as previously described. See, West D W, Kujbida G W, Moore D R, et al. Resistance exercise-induced increases in putative anabolic hormones do not enhance muscle protein synthesis or intracellular signaling in young men. The Journal of physiology 2009; 587(Pt 21):5239-47; and Burd N A, Holwerda A M, Selby K C, et al. Resistance exercise volume affects myofibrillar protein synthesis and anabolic signaling molecule phosphorylation in young men. The Journal of physiology 2010; 588(Pt 16):3119-30. Each biopsy sample was obtained from a separate incision ˜2-3 cm apart. Each participant underwent a total of 6 skeletal muscle biopsies; 3 from each leg. Details of the infusion protocol are outlined in FIG. 1.

Beverage Composition

Study participants were administered nutrient treatments in a double-blinded manner. The macronutrient and amino acid composition of each of the 5 treatments is outlined below in Table 2.

TABLE 2
Amino acid, protein, CHO, and fat content
of the nutritional treatments
Nutritional Treatments

| | W6 + | W6 + | | | W6 + |
|---|---|---|---|---|---|
| | Low-Leu | BCAA | W6 | W25 | High-Leu |
| Endogenous AA | | | | | |
| Alanine, g | 0.29 | 0.29 | 0.29 | 1.15 | 0.29 |
| Arginine, g | 0.13 | 0.13 | 0.13 | 0.53 | 0.13 |
| Aspartic Acid, g | 0.70 | 0.70 | 0.70 | 2.80 | 0.70 |
| Cystine, g | 0.19 | 0.19 | 0.19 | 0.78 | 0.19 |
| Glutamic Acid, g | 1.03 | 1.03 | 1.03 | 4.10 | 1.03 |
| Glycine, g | 0.11 | 0.11 | 0.11 | 0.43 | 0.11 |
| Histidine, g* | 0.14 | 0.14 | 0.14 | 0.55 | 0.14 |
| Isoleucine, g* | 0.34 | 0.34 | 0.34 | 1.35 | 0.34 |
| Leucine, g* | 0.75 | 0.75 | 0.75 | 3.00 | 0.75 |
| Lysine, g* | 0.68 | 0.68 | 0.68 | 2.70 | 0.68 |
| Methionine, g* | 0.14 | 0.14 | 0.14 | 0.58 | 0.14 |
| Phenylalanine, g* | 0.22 | 0.22 | 0.22 | 0.88 | 0.22 |
| Proline, g | 0.26 | 0.26 | 0.26 | 1.05 | 0.26 |
| Serine, g | 0.16 | 0.16 | 0.16 | 0.63 | 0.16 |
| Threonine, g* | 0.28 | 0.28 | 0.28 | 1.10 | 0.28 |
| Tryptophan, g | 0.17 | 0.17 | 0.17 | 0.68 | 0.17 |
| Tyrosine, g | 0.22 | 0.22 | 0.22 | 0.88 | 0.22 |
| Valine, g* | 0.35 | 0.35 | 0.35 | 1.38 | 0.35 |
| Added AA | | | | | |
| Alanine, g | 2.05 | 0.03 | 3.18 | 0.00 | 1.05 |
| Glycine, g | 2.05 | 0.03 | 3.17 | 0.00 | 1.05 |
| Leucine, g* | 2.25 | 4.25 | 0.00 | 0.00 | 4.25 |
| Isoleucine, g* | 0.00 | 1.01 | 0.00 | 0.00 | 0.00 |
| Valine, g* | 0.00 | 1.03 | 0.00 | 0.00 | 0.00 |
| Added CHO, g | 35.0 | 35.0 | 35.0 | 22.60 | 35.0 |
| Added Fat, g | 5.68 | 5.68 | 5.68 | 5.68 | 5.68 |
| Totals | | | | | |
| Whey Protein, g | 6.15 | 6.15 | 6.15 | 24.57 | 6.15 |
| EAA, g* | 5.14 | 9.18 | 2.89 | 11.54 | 7.14 |
| NEAA, g | 7.36 | 3.32 | 9.61 | 13.03 | 5.36 |
| Total Protein, g | 12.50 | 12.50 | 12.50 | 24.57 | 12.50 |
| Leucine, g* | 3.00 | 5.00 | 0.75 | 3.00 | 5.00 |
| Isoleucine, g* | 0.34 | 1.35 | 0.34 | 1.35 | 0.34 |
| Valine, g* | 0.35 | 1.38 | 0.35 | 1.38 | 0.35 |
| BCAA, g* | 3.68 | 7.73 | 1.43 | 5.73 | 5.68 |
| CHO, g | 35.0 | 35.0 | 35.0 | 22.90 | 35.0 |
| Fat, g | 5.68 | 5.68 | 5.68 | 5.68 | 5.68 |
| Kcal | 241.0 | 241.0 | 241.0 | 241.0 | 241.0 |

The W6+Low-Leu, W6+BCAA, W6+High-Leu, and W6 treatments were iso-nitrogenous, iso-energetic, and macronutrient-matched while the positive control (W25) contained a reduced amount of carbohydrate and more protein to be energy-matched to the other treatments. The whey protein isolate was independently tested in triplicate for content analysis. The free-form amino acids used were as follows: L-leucine, L-isoleucine, L-valine, L-alanine, L-glycine. The CHO source was sucrose while the fat source was hydrogenated coconut oil. All nutrient treatments were prepared in 300 mL of water (see, Table 2). To minimize disturbances in isotopic equilibrium following amino acid ingestion, beverages were enriched to 4% with L-[ring-¹³C₆] phenylalanine based on a phenylalanine content of 3.5% in the whey protein.

Analytical Methods

Blood glucose was measured using a blood glucose meter. Blood amino acid concentrations were analyzed via high performance liquid chromatography as described previously. See, Wilkinson S B, Tarnopolsky M A, Macdonald M J, Macdonald J R, Armstrong D, Phillips S M. Consumption of fluid skim milk promotes greater muscle protein accretion after resistance exercise than does consumption of an isonitrogenous and isoenergetic soy-protein beverage. The American journal of clinical nutrition 2007; 85(4):1031-40. Plasma L-[ring-¹³C₆] phenylalanine enrichment was determined as previously described. See, Glover E I, Oates B R, Tang J E, Moore D R, Tarnopolsky M A, Phillips S M. Resistance exercise decreases eIF2Bepsilon phosphorylation and potentiates the feeding-induced stimulation of p70S6K1 and rpS6 in young men. American journal of physiology Regulatory, integrative and comparative physiology 2008; 295(2):R604-10. Plasma insulin concentration was measured using a commercially available immunoassay kit.

Muscle samples (˜40-50 mg) were homogenized on ice in buffer. Samples were then centrifuged at 15,000 g for 10 minutes 4° C. The supernatant was removed and protein concentrations were determined via BCA protein assay. The pellet containing the myofibrillar proteins was stored at −80° C. for future processing. Working samples of equal concentration were prepared in Laemmli buffer. See, Laemmli U K. Cleavage of structural proteins during the assembly of the head of bacteriophage T4. Nature 1970; 227(5259):680-5. Equal amounts (20 μg) of protein were loaded onto 10% or gradient precast gels for separation by electrophoresis. Proteins were then transferred to a polyvinylidene fluoride membrane, blocked (5% skim milk) and incubated overnight at 4° C. in primary antibody: phospho-Akt^{Ser473}, phospho-mTOR^{Ser2448,} phospho-p70 S6 Kinase^{Thr389}, phospho-4E-BP1^{Thr37/46}, phosphor eEF2^{Thr56}, Phospho-S6 Ribosomal protein. Membranes were then washed and incubated in secondary antibody (1 hour at room temperature) before detection with chemiluminescence. Phosphorylation status was expressed relative to a-tubulin and is presented for each protein as fold-change from basal fasted conditions. Images were quantified by spot densitometry using ImageJ software.

Muscle biopsy samples were processed as previously described. See, Moore D R, Tang J E, Burd N A, Rerecich T, Tarnopolsky M A, Phillips S M. Differential stimulation of myofibrillar and sarcoplasmic protein synthesis with protein ingestion at rest and after resistance exercise. The Journal of physiology 2009; 587(Pt 4):897-904. To determine the intracellular ¹³C₆ phenylalanine enrichment, ˜20-25 mg of muscle was homogenized in 0.6 M perchloric acid/L. Free amino acids in the resulting supernatant fluid were then passed over an ion-exchange resin and converted to their heptafluorobutyric derivatives for analysis via gas chromatography-mass spectrometry by monitoring ions 316 and 322 after electron ionization. To determine muscle free intracellular amino acid concentrations, samples were processed as previously described. See, Wilkinson S B, Tarnopolsky M A, Macdonald M J, Macdonald J R, Armstrong D, Phillips S M. Consumption of fluid skim milk promotes greater muscle protein accretion after resistance exercise than does consumption of an isonitrogenous and isoenergetic soy-protein beverage. The American journal of clinical nutrition 2007; 85(4):1031-40. Briefly, muscle samples were derivatized and analyzed by HPLC. To determine myofibrillar protein-bound enrichments, a separate piece (˜40-50 mg) of muscle was homogenized in a standard buffer containing protease and phosphatase inhibitors as described above under ‘Immunoblotting’. The supernatant fluid was collected for Western blot analysis as described above, and the pellet was further processed to extract myofibrillar proteins as previously described. See, Moore D R, Tang J E, Burd N A, Rerecich T, Tarnopolsky M A, Phillips S M. Differential stimulation of myofibrillar and sarcoplasmic protein synthesis with protein ingestion at rest and after resistance exercise. The Journal of physiology 2009; 587(Pt 4):897-904. The resulting myofibrillar ‘enriched’ protein pellet was hydrolyzed in 6 M HCL at 110° overnight. Subsequently, the free amino acids were purified using ion-exchange chromatography and converted to their N-acetyl-n-propyl ester derivatives for analysis by gas chromatography combustion isotope ratio mass spectrometry.

Calculations

The fractional synthetic rate (“FSR”) of myofibrillar protein was calculated using the standard precursor-product equation:

FSR=[(E_{2b}−E_{1b})/(E_{IC}×t)]×100

Where E_{b} is the enrichment of bound (myofibrillar) protein, E_{IC} is the average enrichment of the intracellular free amino acid precursor pool of two muscle biopsies, and t is the tracer incorporation time in hours. The utilization of “tracer naïve” subjects allowed us to use a pre-infusion blood sample (i.e., a mixed plasma protein fraction) as the baseline enrichment (E_{1b}) for calculation of resting (i.e., “Fasted”) FSR. See, Tang J E, Moore D R, Kujbida G W, Tarnopolsky M A, Phillips S M. Ingestion of whey hydrolysate, casein, or soy protein isolate: effects on mixed muscle protein synthesis at rest and following resistance exercise in young men. J Appl Physiol 2009; 107(3):987-92; and Mittendorfer B, Andersen J L, Plomgaard P, et al. Protein synthesis rates in human muscles: neither anatomical location nor fibre-type composition are major determinants. The Journal of physiology 2005; 563(Pt 1):203-11; and Miller B F, Olesen J L, Hansen M, et al. Coordinated collagen and muscle protein synthesis in human patella tendon and quadriceps muscle after exercise. The Journal of physiology 2005; 567(Pt 3):1021-33.

Statistics

Anthropometric measures and strength tests were compared using a one-factor (treatment) ANOVA. Blood glucose, plasma insulin, blood amino acids (leucine, BCAA, EAA) were analyzed using a two-factor (treatment×time) repeated measures ANOVA. Plasma enrichments were analyzed using a two-factor (treatment×time) repeated measures ANOVA and linear regression. Intracellular precursor pool enrichments were analyzed using a two-factor (treatment×time) repeated measures ANOVA for each condition (FED and EX-FED), a two-factor ANOVA (treatment×condition) at each time point (1.5 and 4.5 hours), and linear regression. Intracellular amino acids (leucine, BCAA, EAA) protein phosphorylation, and myofibrillar FSR were analyzed using a three-factor (treatment×time×condition) mixed model ANOVA. Protein phosphorylation is expressed as fold-change from Fasted. A Tukey post-hoc analysis was performed whenever a significant F ratio was found to isolate specific differences. Statistical analyses were performed using SPSS software package. Values are expressed as means±standard error of the mean (SEM). Means were considered to be statistically different for P values<0.05.

Results

Participant Characteristics

Participant characteristics are shown above in Table 1. There were no differences between treatment groups for any of the anthropometric variables examined (all P>0.05).

Exercise Variables

There were no differences between treatment groups for 1-RM (see, Table 1), or the product of load (kg)×volume (# of repetitions) for exercise performed during the experiment (data not shown; all P>0.05).

Dietary Run-In

Participants received ˜1.4-1.5 g protein/kg bodyweight/day during the standardized diet with no differences between treatment groups. There were no differences between treatment groups for total energy, protein, carbohydrate, or fat (data not shown) (all P>0.05).

Blood glucose, plasma insulin, and blood amino acid concentrations. Blood glucose concentration showed a rapid but transient increase following treatment administration demonstrating main effects for time (P=<0.001) and treatment (P=0.019).

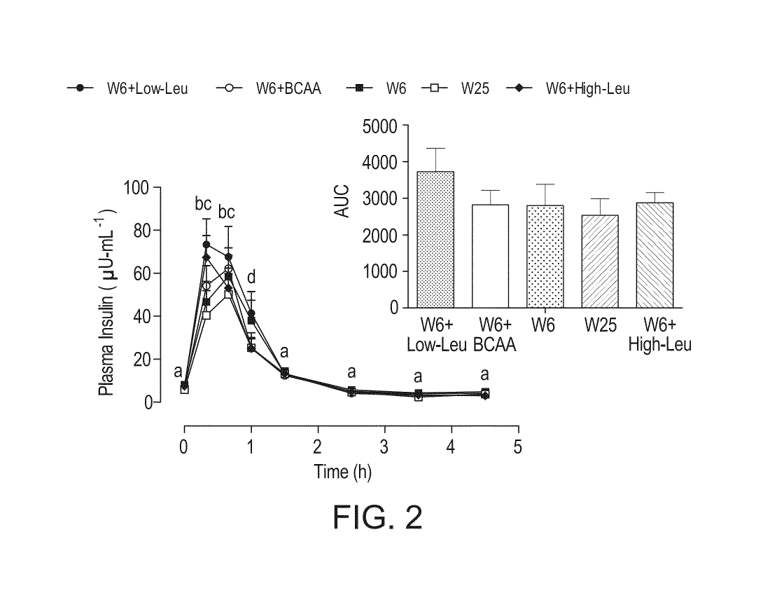

Plasma insulin concentration increased rapidly following treatment administration showing a main effect for time (P<0.001; see, FIG. 2). Area under the insulin curve (AUC inset; see, FIG. 2) following treatment administration was not different between treatment groups (P=0.497).

Concentration over time for blood leucine, isoleucine, valine, and sum of the essential amino acids are shown in panel A-D respectively in FIG. 3. No statistical analysis was performed on the concentration over time data. Area under the curve (“AUC”), maximum concentration (“Cₘₐₓ”), time of maximum concentration (“Tₘₐₓ”), and area under the curve below baseline (“AUC_{neg}”) were analyzed for blood leucine, isoleucine, valine, and ΣEAA and are presented in Table 3 below.

TABLE 3
Variables of blood leucine, isoleucine, valine, and ΣEAA following treatment administration

| | W6 + Low-Leu | W6 + BCAA | W6 | W25 | W6 + High-Leu | |
|---|---|---|---|---|---|---|
| leucine | 12234 ± 1629 | 27517 ± 4493^{‡} | 3223 ± 1465 | 19252 ± 3393^{‡} | 35278 ± 6016*^{‡#} | AUC |
| | 295 ± 40 | 459 ± 75^{‡} | 145 ± 18 | 309 ± 51 | 554 ± 74*^{‡#} | Cₘₐₓ (μmol · l⁻¹) |
| | 43 ± 5 | 51 ± 7 | 49 ± 8 | 75 ± 14 | 41 ± 9 | Tₘₐₓ (min) |
| | −1510 ± 903 | −991 ± 469 | −4654 ± 1906^{#+} | −196 ± 157 | −565 ± 282 | AUC^{neg} |
| isoleucine | 432 ± 142 | 4225 ± 883⁺ | 1145 ± 764 | 6692 ± 1766*^{‡+} | 344 ± 157 | AUC |
| | 55 ± 10 | 122 ± 22 | 61 ± 9 | 131 ± 29*⁺ | 55 ± 8 | Cₘₐₓ (μmol · l⁻¹) |
| | 40 ± 54^{#} | 45 ± 6 | 57 ± 9 | 70 ± 8 | 35 ± 6^{#} | Tₘₐₓ (min) |
| | −3503 ± 581^{#} | −2073 ± 445 | −2919 ± 721 | −643 ± 261 | −5618 ± 1039^{†#} | AUC^{neg} |
| valine | 489 ± 165 | 5347 ± 1373 | 2189 ± 1741 | 7752 ± 2516*⁺ | 665 ± 337 | AUC |
| | 150 ± 14 | 246 ± 40 | 178 ± 21 | 229 ± 44 | 162 ± 16 | Cₘₐₓ (μmol · l⁻¹) |
| | 40 ± 5 | 58 ± 10 | 53 ± 10 | 74 ± 10 | 33 ± 5^{#} | Tₘₐₓ (min) |
| | −9457 ± 1689 | −5247 ± 2163 | −9690 ± 2451 | −2174 ± 486 | −13988 ± 2012^{†#} | AUC^{neg} |
| ΣEAA | 11841 ± 2310 | 48937 ± 11917 | 11739 ± 6425 | 62722 ± 18780*^{‡} | 31047 ± 7267 | AUC |
| | 801 ± 95 | 1183 ± 175 | 742 ± 85 | 1175 ± 188 | 1088 ± 90 | Cₘₐₓ (μmol · l⁻¹) |
| | 40 ± 5^{#} | 56 ± 7 | 53 ± 10 | 83 ± 17 | 39 ± 9^{#} | Tₘₐₓ (min) |
| | −18734 ± 4681 | −6135 ± 2122 | −22492 ± 9420 | −3028 ± 1282 | −16198 ± 5315 | AUC^{neg} |

Both AUC and Cₘₐₓ for blood leucine were greatest following W6+High-Leu being statistically different from W6+Low-Leu, W6, and W25. For both isoleucine and valine, AUC_{neg} was reduced following W6+BCAA and W25, being statistically different from W6+High-Leu. Time of maximum concentration (“Tₘₐₓ”) for leucine, isoleucine, valine, and ΣEAA tended to occur latest for W25 and most rapidly for W6+High-Leu (see, Table 3).

Intracellular Leucine, Isoleucine, Valine, and ΣEAA

Intracellular concentrations of leucine, isoleucine, valine and ΣEAA are shown in Table 4 below.

TABLE 4
Intracellular concentrations (μM) of leucine, isoleucine, valine,
and ΣEAA following treatment administration

| | W6 + Low-Leu | W6 + BCAA | W6 | W25 | W6 + High-Leu | |
|---|---|---|---|---|---|---|
| leucine | 209 ± 15^{a} | 229 ± 17^{a} | 214 ± 11^{a} | 212 ± 16^{a} | 222 ± 18^{a} | Fasted |
| | 248 ± 16^{b} | 313 ± 24^{‡b} | 215 ± 18^{a} | 274 ± 32^{b} | 344 ± 16*^{‡#b} | 1.5 h FED |
| | 221 ± 14^{a} | 232 ± 12^{a} | 210 ± 8^{a} | 248 ± 8^{‡a} | 263 ± 27*^{‡a} | 4.5 h FED |
| | 267 ± 12^{b} | 299 ± 36^{‡b} | 222 ± 19^{a} | 255 ± 23^{b} | 351 ± 17*^{‡#b} | 1.5 h EX-FED |
| | 205 ± 20^{a} | 225 ± 15^{a} | 188 ± 14^{a} | 224 ± 17^{‡a} | 249 ± 31*^{‡a} | 4.5 h EX-FED |
| isoleucine | 254 ± 33 | 296 ± 38 | 216 ± 27 | 322 ± 41 | 290 ± 37 | Fasted^{a} |
| | 294 ± 65 | 389 ± 67 | 219 ± 32 | 365 ± 47 | 312 ± 46 | 1.5 h FED^{b} |
| | 276 ± 64 | 297 ± 52 | 208 ± 36 | 333 ± 39 | 261 ± 50 | 4.5 h FED^{a} |
| | 299 ± 64 | 321 ± 44 | 218 ± 44 | 340 ± 38 | 284 ± 56 | 1.5 h EX-FED^{a} |
| | 311 ± 76 | 348 ± 71 | 215 ± 57 | 330 ± 30 | 302 ± 65 | 4.5 h EX-FED^{a} |
| valine | 280 ± 26 | 261 ± 16 | 232 ± 23 | 259 ± 24 | 288 ± 25 | Fasted^{a} |
| | 224 ± 9 | 262 ± 15 | 252 ± 27 | 280 ± 29 | 308 ± 22 | 1.5 h FED^{a} |
| | 216 ± 14 | 240 ± 17 | 222 ± 16 | 295 ± 6 | 195 ± 16 | 4.5 h FED^{b} |
| | 250 ± 13 | 248 ± 21 | 246 ± 21 | 274 ± 30 | 255 ± 33 | 1.5 h EX-FED^{a} |
| | 212 ± 16 | 246 ± 25 | 208 ± 27 | 264 ± 26 | 232 ± 33 | 4.5 h EX-FED^{b} |
| ΣEAA | 2730 ± 157 | 3142 ± 273 | 2966 ± 185 | 2718 ± 217 | 3016 ± 229 | Fasted^{a} |
| | 2837 ± 201 | 2946 ± 134 | 3336 ± 366 | 3118 ± 288 | 3212 ± 269 | 1.5 h FED^{a} |
| | 2801 ± 176 | 2741 ± 115 | 3301 ± 256 | 2925 ± 136 | 2818 ± 350 | 4.5 h FED^{a} |
| | 3110 ± 251 | 2732 ± 277 | 3190 ± 171 | 2684 ± 212 | 2626 ± 149 | 1.5 h EX-FED^{a} |
| | 2657 ± 273 | 3066 ± 181 | 3163 ± 277 | 3198 ± 290 | 3261 ± 359 | 4.5 h EX-FED^{a} |

Intracellular leucine demonstrated a time×treatment interaction (P=0.031), increasing at 1.5 hours post-treatment for all treatment groups except W6, but returning to values not different from Fasted by 4.5 hours. Intracellular isoleucine demonstrated a time×condition interaction (P=0.012) increasing above Fasted at 1.5 hours in the FED condition only. Intracellular valine demonstrated a main effect for time (P=0.006) falling below basal Fasted concentrations at 4.5 hours. There were no time (P=0.691), treatment (P=0.661), or condition (P=0.707) effects for ΣEAA.

Plasma and Intracellular Free Phenylalanine Enrichments

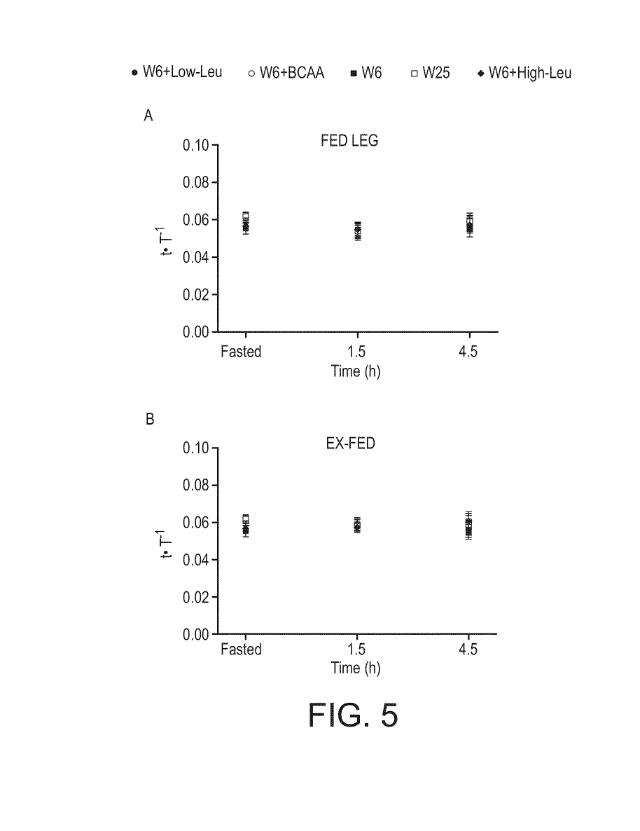

Intracellular free phenylalanine enrichments were not different between treatments or across time in either FED (Treatment: P=0.926; Time: P=0.124) or EX-FED (Treatment: P=0.334; Time: P=0.945) conditions. Further, there were no differences between conditions at 1.5 hours (P=0.055) or 4.5 hours (P=0.317). The slope of the intracellular free phenylalanine enrichments were not different from zero for any of the treatment groups in either FED or EX-FED condition (see, FIG. 5).

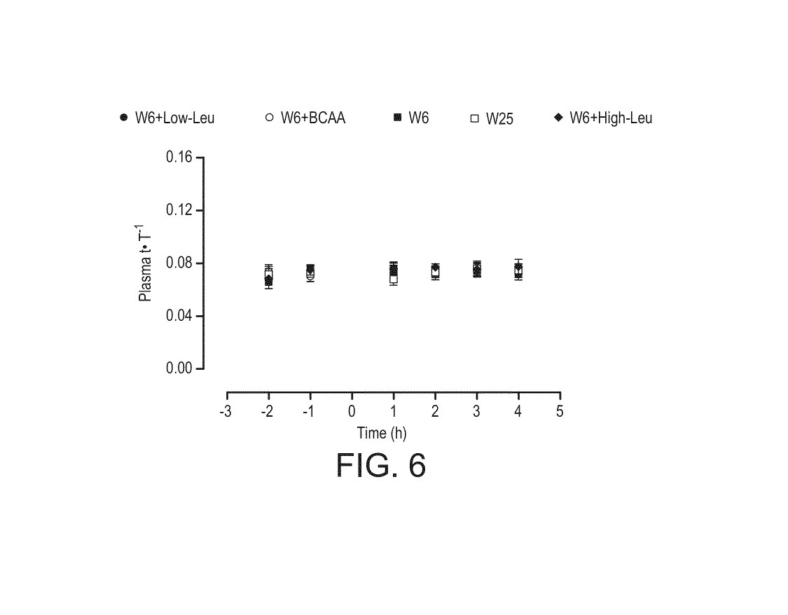

Plasma free phenylalanine enrichments did not differ between treatment groups (P=0.917) or across time (P=0.58). The slope of the plasma free phenylalanine enrichments were not different from zero for any treatment group (see, FIG. 6).

Myofibrillar Protein Synthesis

Myofibrillar fractional synthesis rates (“FSR”) rates are shown in FIG. 4. Myofibrillar FSR demonstrated a treatment×time interaction (P=0.002) whereby FSR rates were increased compared with basal (“Fasted”) in all treatment groups when measured over 0-1.5 hours. Over 1.5-4.5 hours post-exercise, FSR rates remained increased compared to Fasted in all treatment groups; however, FSR rates in W25 and W6+High-Leu were greater than W6+Low-Leu, W6+BCAA, and W6. There were no statistical differences between FED and EX-FED conditions (P=0.483).

Muscle Anabolic Signaling

Changes in the phosphorylation status of signaling proteins involved in the regulation of mRNA translation initiation and elongation are shown in Table 5 below.

TABLE 5
Western-blot analysis of protein synthesis-associated signaling proteins following treatment administration

| | W6 + Low-Leu | W6 + BCAA | W6 | W25 | W6 + High-Leu | |
|---|---|---|---|---|---|---|
| | Fold difference from baseline^{a} | | | | | |
| p-Akt^{(Ser 473)} | 2.89 ± 0.69^{†‡b} | 1.52 ± 0.25^{a} | 1.61 ± 0.30^{a} | 2.09 ± 0.38^{b} | 1.76 ± 0.18^{a} | 1.5 h FED |
| | 1.67 ± 0.40^{†‡+$a} | 0.71 ± 0.16^{a} | 0.70 ± 0.17^{a} | 1.01 ± 0.24^{a} | 0.74 ± 0.09^{a} | 4.5 h FED |
| | 4.23 ± 1.40^{‡+$b} | 2.47 ± 0.24^{a} | 1.45 ± 0.18^{a} | 3.69 ± 0.64^{‡$b} | 2.03 ± 0.46^{a} | 1.5 h EX-FED |
| | 1.07 ± 0.10^{a} | 1.20 ± 0.16^{a} | 1.38 ± 0.41^{$a} | 1.50 ± 0.30^{a} | 1.17 ± 0.21^{a} | 4.5 h EX-FED |
| p-mTOR^{(Ser 2448)} | 1.59 ± 0.49^{b} | 1.35 ± 0.15^{a} | 1.05 ± 0.15^{a} | 1.52 ± 0.29^{b} | 1.89 ± 0.31^{†‡b} | 1.5 h FED |
| | 0.86 ± 0.19^{a} | 0.89 ± 0.15^{a} | 0.62 ± 0.15^{a} | 1.25 ± 0.22^{a} | 1.63 ± 0.26^{†‡b} | 4.5 h FED |
| | 1.45 ± 0.29^{b} | 1.45 ± 0.14^{a} | 1.23 ± 0.16^{a} | 1.60 ± 0.09^{b} | 2.14 ± 0.36^{†‡b} | 1.5 h EX-FED |
| | 1.41 ± 0.39^{a} | 1.11 ± 0.14^{a} | 0.87 ± 0.11^{a} | 1.10 ± 0.23^{a} | 1.71 ± 0.25^{†‡b} | 4.5 h EX-FED |
| p-p70S6K1^{(Thr 389)} | 1.13 ± 0.09 | 0.98 ± 0.08 | 1.04 ± 0.10 | 1.02 ± 0.07 | 1.12 ± 0.13 | 1.5 h FED^{a} |
| | 1.02 ± 0.11 | 0.86 ± 0.12 | 0.97 ± 0.13 | 0.97 ± 0.13 | 1.14 ± 0.14 | 4.5 h FED^{a} |
| | 1.13 ± 0.09 | 0.92 ± 0.10 | 1.15 ± 0.11 | 1.06 ± 0.06 | 1.07 ± 0.11 | 1.5 h EX-FED^{a} |
| | 1.06 ± 0.08 | 0.93 ± 0.09 | 1.01 ± 0.12 | 1.02 ± 0.10 | 1.33 ± 0.09 | 4.5 h EX-FED^{a} |
| p-4E-BP1^{(Thr 37/46)} | 1.19 ± 0.12 | 1.20 ± 0.12 | 1.02 ± 0.12 | 1.25 ± 0.18 | 1.58 ± 0.24 | 1.5 h FED^{b} |
| | 0.95 ± 0.15 | 1.04 ± 0.19 | 0.73 ± 0.07 | 0.86 ± 0.17 | 0.85 ± 0.14 | 4.5 h FED^{a} |
| | 1.28 ± 0.16 | 1.38 ± 0.20 | 1.05 ± 0.17 | 1.77 ± 0.48 | 1.59 ± 0.38 | 1.5 h EX-FED^{b} |
| | 1.05 ± 0.18 | 1.12 ± 0.10 | 1.02 ± 0.18 | 1.20 ± 0.29 | 1.45 ± 0.22 | 4.5 h EX-FED^{$a} |
| p-rpS6^{(Ser 240/244)} | 2.46 ± 0.93 | 2.66 ± 0.65 | 1.72 ± 0.41 | 1.93 ± 0.32 | 2.45 ± 0.56 | 1.5 h FED^{b} |
| | 2.31 ± 0.59 | 1.63 ± 0.32 | 1.31 ± 0.22 | 2.09 ± 0.42 | 1.12 ± 0.30 | 4.5 h FED^{b} |
| | 3.34 ± 0.93 | 3.67 ± 1.27 | 3.25 ± 1.08 | 4.45 ± 1.63 | 4.49 ± 1.48 | 1.5 h EX-FED^{$b} |
| | 2.86 ± 1.26 | 2.41 ± 0.65 | 1.47 ± 0.42 | 1.38 ± 0.15 | 2.44 ± 0.59 | 4.5 h EX-FED^{b} |
| p-eEF2^{(Thr 56)} | 0.96 ± 0.05 | 1.11 ± 0.09 | 0.95 ± 0.06 | 1.02 ± 0.06 | 1.08 ± 0.04 | 1.5 h FED^{a} |
| | 1.08 ± 0.13 | 0.99 ± 0.07 | 0.93 ± 0.07 | 1.12 ± 0.12 | 1.00 ± 0.06 | 4.5 h FED^{a} |
| | 1.15 ± 0.08 | 1.09 ± 0.05 | 0.92 ± 0.03 | 1.12 ± 0.10 | 1.13 ± 0.04 | 1.5 h EX-FED^{a} |
| | 1.11 ± 0.10 | 1.07 ± 0.11 | 0.88 ± 0.06 | 1.03 ± 0.07 | 1.05 ± 0.04 | 4.5 h EX-FED^{a} |

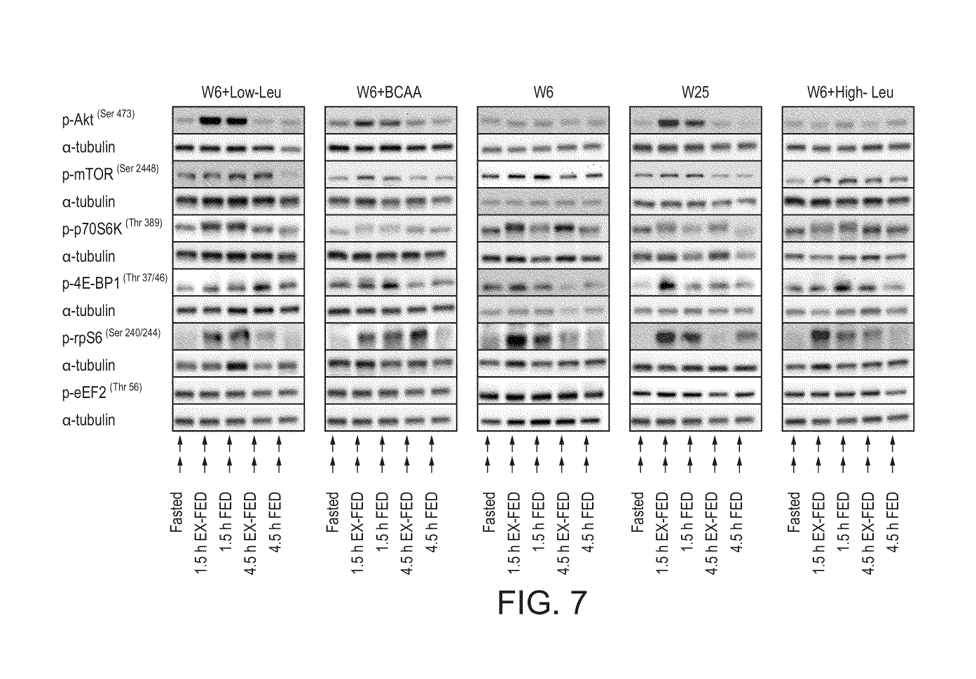

Protein kinase B (p-Akt_{Ser473}) showed a treatment×time×condition interaction (P=0.025). p-mTOR^{Ser2448} showed a treatment×time interaction (P=0.041) whereby at 1.5 hours, p-mTOR^{Ser2448} was increased following W6+Low-Leu, W25, and W6+High-Leu. At 4.5 hours, p-mTOR^{Ser2448} remained increased following W6+High-Leu, with p-mTOR^{Ser2448}. p-p70S6k^{Thr 389} showed no effect of time (P=0.377), treatment (P=0.353), or condition (P=0.062) at the times examined p-4E-BP1^{Thr 37/46} showed a condition×time interaction (P=0.044) whereby both conditions (FED and EX-FED) were increased above Fasted at 1.5 hours, while at 4.5 hours, p-4E-BP1^{Thr 37/46} was significantly greater in the EX-FED vs. FED condition. p-rps6^{Ser 240/244} showed a condition×time interaction (P<0.001) whereby both conditions (FED and EX-FED) were increased above Fasted at both 1.5 hours and 4.5 hours; however the increase in the EX-FED condition at 1.5 hours was greater than the FED condition. p-eEF2^{Thr 56} showed no effect of time (P=0.197), treatment (P=0.384), or condition (P=0.091) at the times examined Representative blot images are shown in FIG. 7.

### Example 2

### Materials and Methods

Ethical Approval

All participants provided written informed consent before initiation the study, approved by the Central Regional Ethics Committee of New Zealand.

Participants

Twelve endurance-trained male cyclists with mean (SD) age 30 y (7), stature 179 cm (5), and weight 78.1 kg (7.8) completed the study. VO_{2 max} was 60.4 mL·kg⁻¹·min⁻¹ (6.2) with corresponding Wₘₐₓ of 323 W (32).

Design

The research design was a single-blind crossover with each subject completing three randomized 1-week experimental periods with exercise and diet control, interspersed with a 1-week washout. Tested were the effects of high (15LEU) and moderate (5LEU) protein-leucine beverages against a control isocaloric to 15LEU, but with zero protein and leucine. Specifically, twelve trained men completed 100 minutes of high-intensity cycling, then ingested 70/15/180/30 g protein/leucine/carbohydrate/fat (“15LEU”), 23/5/180/30 g (“5LEU”) or 0/0/274/30 g (“CON”) beverages in 4 servings during the first 90 minutes of a 240-minute monitored recovery period. Outcome measures were obtained from blood and skeletal muscle tissue collected following intense cycling. Myofibrillar FSR was determined by [ring-¹³C₆]phenylalanine infusion with vastus-lateralis biopsies taken 30 and 240-minutes into recovery.

Preliminary Testing, Diet and Activity

Two weeks prior to the first experimental period, cyclists completed a standard test to determine VO_{2 max} and Wₘₐₓ. See, Nelson A R, Phillips S M, Stellingwerff T, Rezzi S, Bruce S J, Breton I, Thorimbert A, Guy P A, Clarke J, Broadbent S & Rowlands D S. (2012). A protein-leucine supplement increases BCAA and nitrogen turnover but not performance. Med Sci Sports Exerc 44, 57-68. The next day participants completed a familiarization ride of the experimental exercise protocol. Physical activity and diet were standardized for 5-days prior to each experimental period. Exercise was controlled 2-days prior and consisted of a 90-minute ride on an ergometer comprising warm up of 10 minutes at 30% (Wₘₐₓ), 8 minutes at 40%, 2 minutes at 50%, then intervals (4×5 minutes at 70%) interspersed with three blocks of 3×2-minute intervals at 85%, 80%, and 75%, respectively, interspersed with 2-minute periods at 50%, followed by 5 min at 40%. Following this ride and for the remainder of day and day following, participants performed no training and were provided with a pre-weighed diet providing sufficient energy to balance individual caloric requirements based on the Harris-Benedict equation for activity factor of 1.6 (14.9±1.5 MJ·d⁻¹; 58% carbohydrate, 13% protein and 29% fat). On the testing day, participants ingested their final meal 3-hours prior to reporting to the laboratory at 1500 hours.

Experimental Protocol

A 20-gauge catheter with stopcock was positioned in the antecubital vein of each arm for infusion and sampling. The line was rendered patent with isotonic saline. One catheter was attached to syringe pump, the second was for sampling. A baseline blood sample was taken prior to commencing a primed constant infusion of L-[ring-¹³C₆] phenylalanine (99% enriched) at a rate of 0.5 μmol·kg⁻¹·min⁻¹ (prime; 2 μmol·kg⁻¹·min⁻¹) beginning 10 minutes into exercise. The exercise protocol totaled 100 minutes and comprised: a warm-up, as above; intervals (% Wₘₐₓ) of 8×2-minutes (90%), 2×5 minutes (70%), 2×2 minutes (80%) and 3×1 minute (100%), interspersed with recovery 2-minutes (50%); and 8 minutes cool-down (40%). During exercise participants consumed 800 ml·h⁻¹ of artificially sweetened electrolyte solution to maintain hydration and were fan cooled. Following exercise, participants showered, and then ingested the first nutrition serving 10-minutes after cessation of exercise and subsequently every 30 minutes over the first 90 minutes of the 240-minutes assessed recovery (4 serves). Participant's rested semi-supine during recovery. Muscle biopsies were collected at 30 minutes and 240 minutes into recovery from the vastus lateralis as described previously. See, Fu M H, Maher A C, Hamadeh M J, Ye C & Tarnopolsky M A. (2009). Exercise, sex, menstrual cycle phase, and 17 {beta}-estradiol influence metabolism-related genes in human skeletal muscle. Physiol Genomics 40, 34-47.

Intervention and Control Nutrition

The test beverages consisted of milk-like drinks containing milk protein concentrate (MPC 470) and whey protein isolate (WPI 894) (2:1 w/w), L-leucine, maltodextrin and fructose (1:1 w/w), and freeze dried canola oil. Beverages were made up to 1200 mL using water, and split into 4 equal servings. Total 90-minutes intake of protein, leucine, carbohydrate and fat was based on the quantity shown recently to enhance the performance of subsequent intense cycling (see, Thomson J S, Ali A & Rowlands D S. (2011). Leucine-protein supplemented recovery feeding enhances subsequent cycling performance. Appl Physiol Nutr Metabol 36, 242-253) and comprised respectively: 15LEU dose 70/15/180/30 g. The 15LEU dose was compared to ⅓ quantity 23.3/5/180/30 g (5LEU dose), which was an intake hypothesised to maximally saturate myofibrillar FSR, and to the control, CON, 0/0/274/30 g. All beverages also contained 1.4 g NaCl, 14.4 g vanilla essence, and 3.6 g of emulsifier per 1200 mL. L-[ring-¹³C₆] phenylalanine representing 8% of the total ingested amino acid within the protein was added to each 5LEU and 15LEU beverage (0.020 and 0.060 g, respectively) to maintain steady state plasma enrichment.

Sampling and Analysis of Blood Parameters

Blood samples for were taken prior to infusion priming, immediately following exercise and at 15, 30, 60, 90, 120, 180, and 240 minutes into recovery. Blood was treated as described previously for measurement of plasma phenylalanine enrichment, glucose, lactate, amino-acid, and serum insulin concentrations. See, Moore D R, Robinson M J, Fry J L, Tang J E, Glover E I, Wilkinson S B, Prior T, Tarnopolsky M A & Phillips S M. (2009a). Ingested protein dose response of muscle and albumin protein synthesis after resistance exercise in young men. Am J Clin Nutr 89, 161-168; and Moore D R, Tang J E, Burd N A, Rerecich T, Tarnopolsky M A & Phillips S M. (2009b). Differential stimulation of myofibrillar and sarcoplasmic protein synthesis with protein ingestion at rest and after resistance exercise. J Physiol 587, 897-904.

Immunoblots

mTOR-related signaling pathway activation was determined by immunoblot as previously described (see, Lau P, Nixon S J, Parton R G & Muscat G E. (2004). RORa regulates the expression of genes involved in lipid homeostasis in skeletal muscle cells. J Biol Chem 279, 36828-36840) using antibodies from Cell Signaling Technology for AMPKα^{Thr172} (#2531, 1:1000) AMPKα (#2532, 1:2000), mTORC1 (#2972, 1:1000); mTORC1^{Ser2448}, (#2971, 1:1000), p70S6K (#9202, 1:1000), p70S6K^{Thr389} (#9234, 1:1000), 4E-BP1 (#9452, 1:3000), and P-rps6^{Ser240/244} (#2215s, 1:2000), SIRT1 (#2493, 1:2000), P-eEF2^{Thr56} (#2331S, 1:1000), and from Novus Biologicals (Littleton, Colo., USA) for eEF2 (NB100-79934, 1:50,000); α-tubulin was the loading control (#T5168, 1: 4000, Sigma, St. Louis, Mo., USA).

Skeletal Muscle Protein Fractional Synthesis

Myofibrillar and mitochondrial protein fractions were isolated from a piece of wet muscle (˜80 mg). See, Coffey V G, Moore D R, Burd N A, Rerecich T, Stellingwerff T, Garnham A P, Phillips S M & Hawley J A. (2010). Nutrient provision increases signaling and protein synthesis in human skeletal muscle after repeated sprints. Eur J Appl Physiol 111, 1473-1483. Plasma and intracellular amino acids were isolated, derivatized and analyzed for L-[ring-¹³C₆] phenylalanine enrichment by gas chromatography-mass spectrometry, as described previously. See, Moore D R, Tang J E, Burd N A, Rerecich T, Tarnopolsky M A & Phillips S M. (2009b). Differential stimulation of myofibrillar and sarcoplasmic protein synthesis with protein ingestion at rest and after resistance exercise. J Physiol 587, 897-904. Gas chromatography-combustion isotope ratio mass spectrometry was used to determine muscle protein enrichment, with myofibrillar FSR calculated using the standard precursor-product method, as previously described. See, Id. There was insufficient tissue to complete analysis of mitochondrial FSR.

Illumina Microarray

RNA extraction, labeled-cRNA synthesis, and hybridization selection were performed as described previously. See, Rowlands D S, Thomson J S, Timmons B W, Raymond F, Fuerholz A, Mansourian R, Zawhlen M C, Metairon S, Glover E, Stellingwerff T, Kussmann M & Tarnopolsky M A. (2011). The transcriptome and translational signaling following endurance exercise in trained skeletal muscle: impact of dietary protein. Physiol Genomics 43, 1004-1020. All samples were analyzed with HumanHT-12 v3.0 Expression BeadChips. The microarray output was deposited online (http://www.ncbi.nlm.nih.gov/geo/, accession number GSE44818). Normalization, transformation, and statistical analysis was conducted as described previously. See, Id. A traditional null-hypothesis based gene selection criteria (ROBP<0.001) was used for probe selection for bioinformatic interrogation. Expression magnitude was defined by fold change and the modified standardized difference (effect size). See, Id. The analysis returned sufficient power to detect a large standardized difference of 1.3. Gene selections were interrogated within Ingenuity Pathway Analysis software.

Confirmatory Gene Expression

The NanoString nCounter gene expression assay was used to confirm the magnitude of top-ranked network differential expression gene expression (>1.3-fold; ROBP<0.001) estimated by microarray. Briefly, 750 ng of RNA in 5 μl was hybridized at 65° C. overnight with the Nanostring Codeset followed by expression quantification. Background correction was by the geNorm-method. See, Vandesompele J, De Preter K, Pattyn F, Poppe B, Van Roy N, De Paepe A & Speleman F. (2002). Accurate normalization of real-time quantitative RT-PCR data by geometric averaging of multiple internal control genes. Genome Biol 3, 18.

Statistical Analysis

The effects of treatment and time on all other dependent variables were estimated from mixed model ANOVA, while the strength of relationships was by linear regression (SAS 9.1; SAS, Cary, N.C.). Where appropriate, data were log transformed prior to analysis. Uncertainty was the 90% confidence interval. Magnitude-based inference and probability were inferred from the standardized difference and the correlation coefficient for linear regressions using the effect size and likelihood thresholds of Hopkins et al. (see, Hopkins W G, Marshall S W, Batterham A M & Hanin J. (2009). Progressive statistics for studies in sports medicine and exercise science. Med Sci Sports Exerc 41, 3-13); the threshold for bioequivalence for FSR was the 80%-125% pharmacokinetic criteria with zero overlap of the 90% confidence interval (see, Committee for Proprietary Medicinal Products & Products EAftEoM. (2001). Note for Guidance on the Investigation of Bioavailability and Bioequivalence).

Results

Myofibrillar Protein Fractional Synthetic Rate

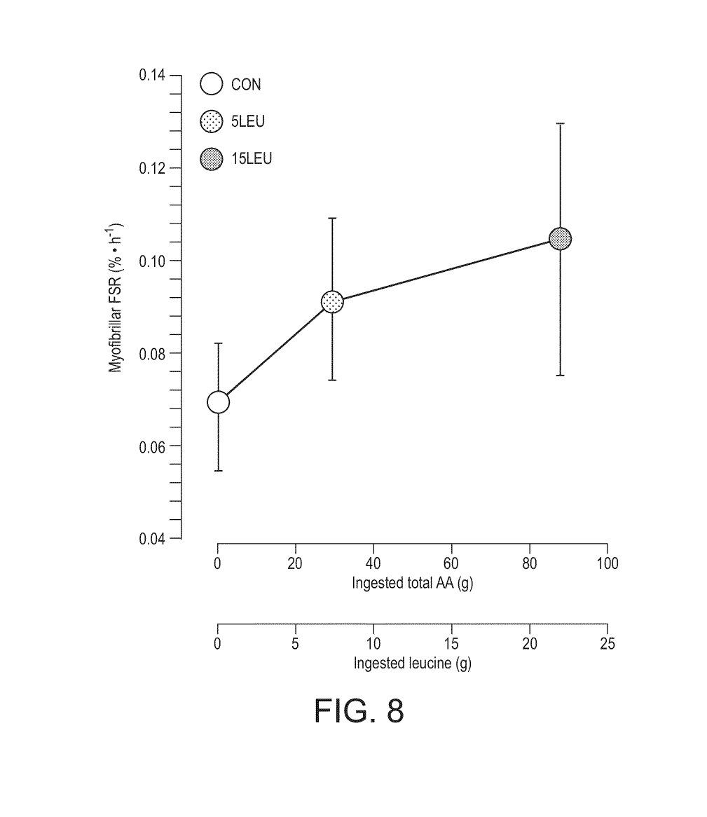

Relative to the pharmacokinetic bioequivalence threshold reference of 25%, the large standardized increase in FSR with 15LEU (51% 90% CL±12%, p=1E-05) and moderate increase with 5LEU vs CON (33%±12%, moderate, 4.6E-05) were clear, but the small increase with 15LEU vs 5LEU was likely bioequivalent (13%±12%, p=0.07). See, FIG. 8.

Plasma Amino Acid Concentrations

Plasma amino-acid concentrations increased in positive relation to ingested protein-leucine quantity. See, FIG. 9. Increases in modeled-overall mean plasma leucine and essential amino-acid concentrations with 15LEU were extremely large (2.8- to 8.0-fold; 90% CL×/÷1.3 to 1.4) vs CON and moderate-large (1.7- to 2.2-fold; ×/÷1.2 to 1.3) vs 5LEU; 5LEU vs CON increased concentrations 1.6- to 3.7-fold (×/÷1.3 to 1.4) (p<0.001 for all contrasts). The increase in total plasma amino-acid concentration was small with 15LEU vs 5LEU (1.3-fold; ×/÷1.3-fold, p=0.06) and vs CON (1.4-fold; ×/÷1.3-fold, p=0.03), but 5LEU vs CON was unclear.

mTORC1 Pathway

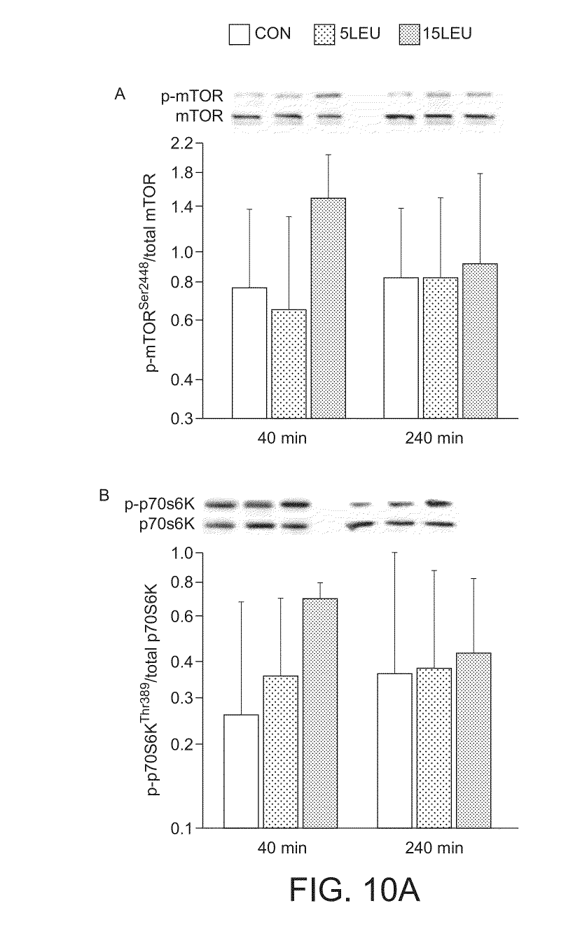

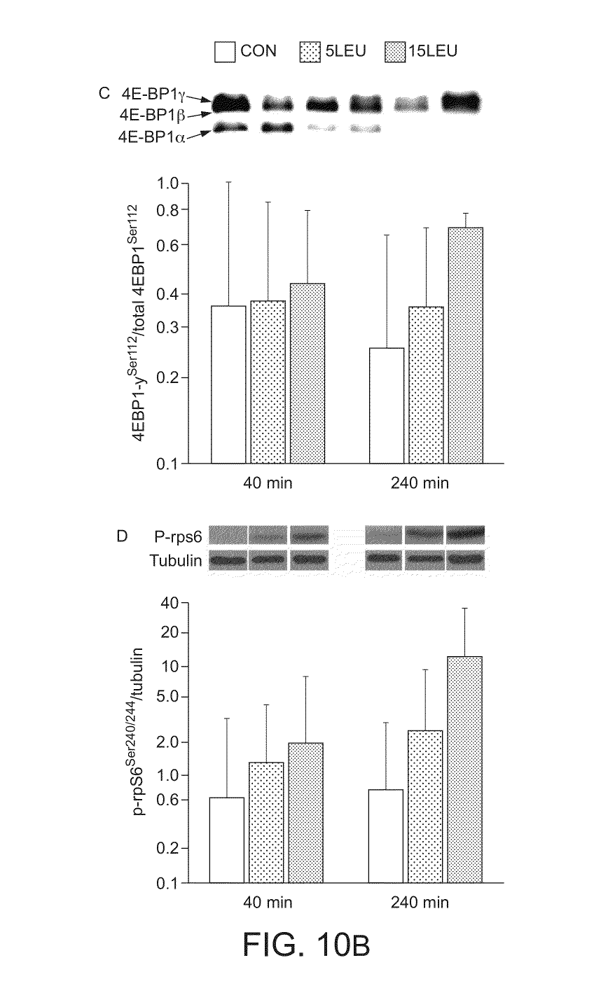

At 30 minutes, 15LEU moderately increased mTOR^{Ser2448} (2.2-fold×/÷1.6-fold, p=0.008) and p70S6K^{Thr389} (3.5-fold×/÷1.9-fold, p=0.003) phosphorylation vs 5LEU, and vs CON (1.9-fold×/÷1.6-fold, p=0.03; 6.7-fold×/÷1.9-fold, p=5.2E-05, respectively). See, FIG. 10. The increase in rpS6^{Ser246/244} phosphorylation with 15LEU (3.2-fold×/÷1.4-fold, p=1.6E-08) and 5LEU (2.1-fold×/÷1.4-fold, p=1.1E-04) vs CON was small. By 240 minutes, there was no clear effect of 15LEU or 5LEU vs CON on mTOR^{Ser2448} phosphorylation, and the increase in p70S6K^{Thr389} phosphorylation with 15LEU vs CON was small (2.0-fold×/÷1.9-fold, p=0.09). In contrast, the increase in rpS6^{Ser246/244} with 15LEU vs CON was extremely large (16.2-fold×/÷1.4-fold, p=4E-28), but the 5LEU vs CON and 15LEU-5LEU dose increases moderate (3.2-fold×/÷1.4-fold, p=1.1E-04 and 4.9-fold×/÷1.4-fold, p=2E-3, respectively). Hyperphosphorylation of 4E-BP1γ was unclear at 30 minutes, but at 240 minutes, increases were moderate with 15LEU (2.5-fold×/÷1.6-fold, p=0.003) and small with 5LEU (2.0-fold×/÷1.6-fold, p=0.02) vs CON.

The effect of treatments on AMPKα^{Thr172} and 4E-BP1^{Thr37/46} phosphorylation were unclear. At 240 minutes there were possible and likely small increases in eEF2^{Thr56}phosphorylation with 15LEU (1.15-fold×/÷1.25-fold, p=0.311) and 5LEU feeding (1.30-fold×/÷1.25-fold, p=0.049) vs CON, respectively. Meanwhile, there was a small increase in total SIRT1 protein with 15LEU vs 5LEU contrast (1.4×/÷1.3-fold, p=2E-3) at 30 minutes but otherwise the effects were trivial.

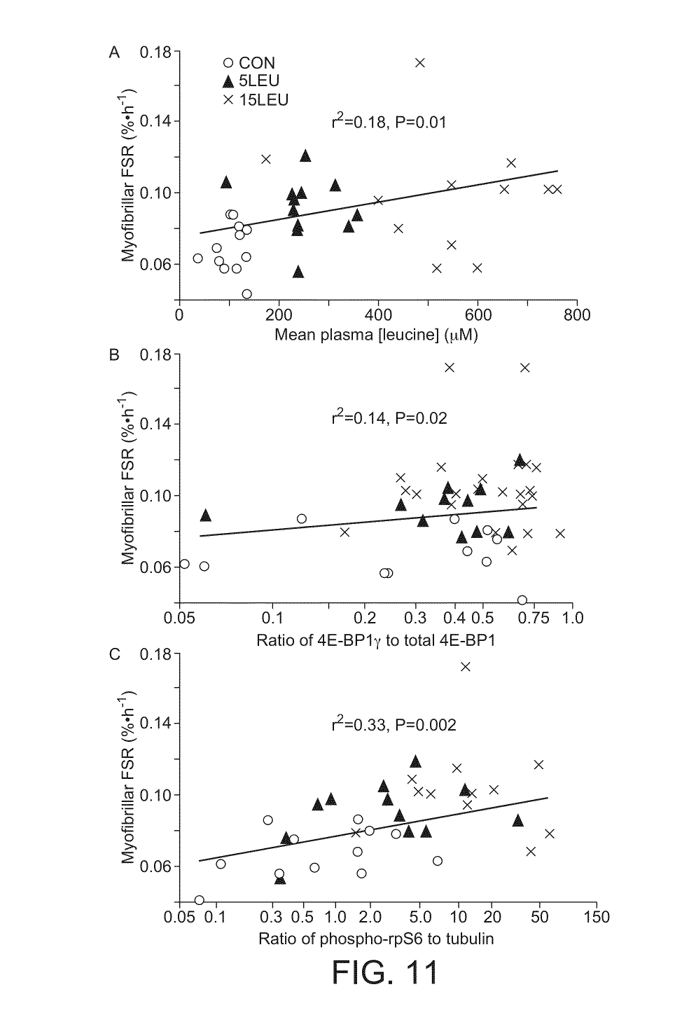

Correlated relationships between myofibrillar FSR, plasma amino acids, and mTORC1 pathway phosphoprotein status are presented in FIG. 11. Briefly, moderate predictors of myofibrillar FSR were p70S6K^{Thr389} and rpS6 phosphorylation, while 4E-BP1γ hyper-phosphorylation, plasma leucine, isoleucine and essential amino acids correlations were small. The correlation between plasma leucine concentration and p70S6K^{Thr389} was small, but moderate against 4E-BP1γ and rpS6 (SM1). Based on regression, from a baseline of 125 μM, a 210 μM increase in mean recovery plasma leucine concentration increased myofibrillar FSR by 0.010%·h⁻¹.

Serum Glucose and Insulin

The overall elevation in mean plasma insulin concentration with 15LEU was small vs CON (60%±20%, p=14E-6) and vs 5LEU (40% 90% CL±20%, p=45E-5), with the 5LEU vs CON difference, trivial. The overall reduction in plasma-glucose concentration with 15LEU was moderate vs CON (−32%±6%, p=2E-12) and small vs 5LEU (−12%±6%, p=0.001).

Nutrient Responsive Transcriptome

Protein-leucine ingestion differentially affected 173-479 genes with ROBP<0.001.

Ingenuity Functions Analysis

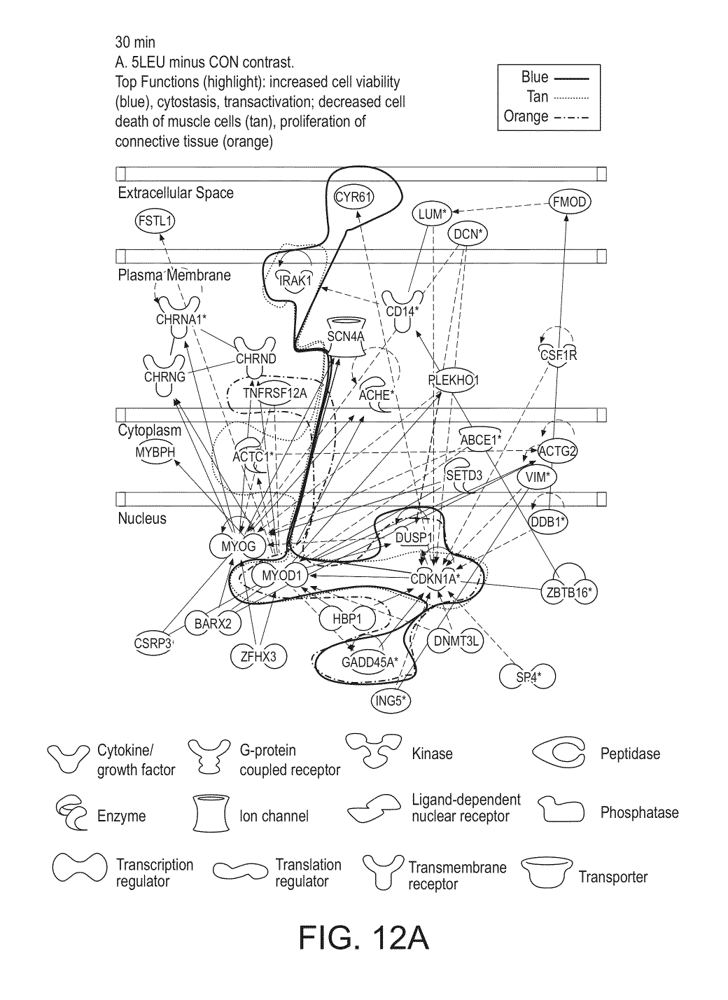

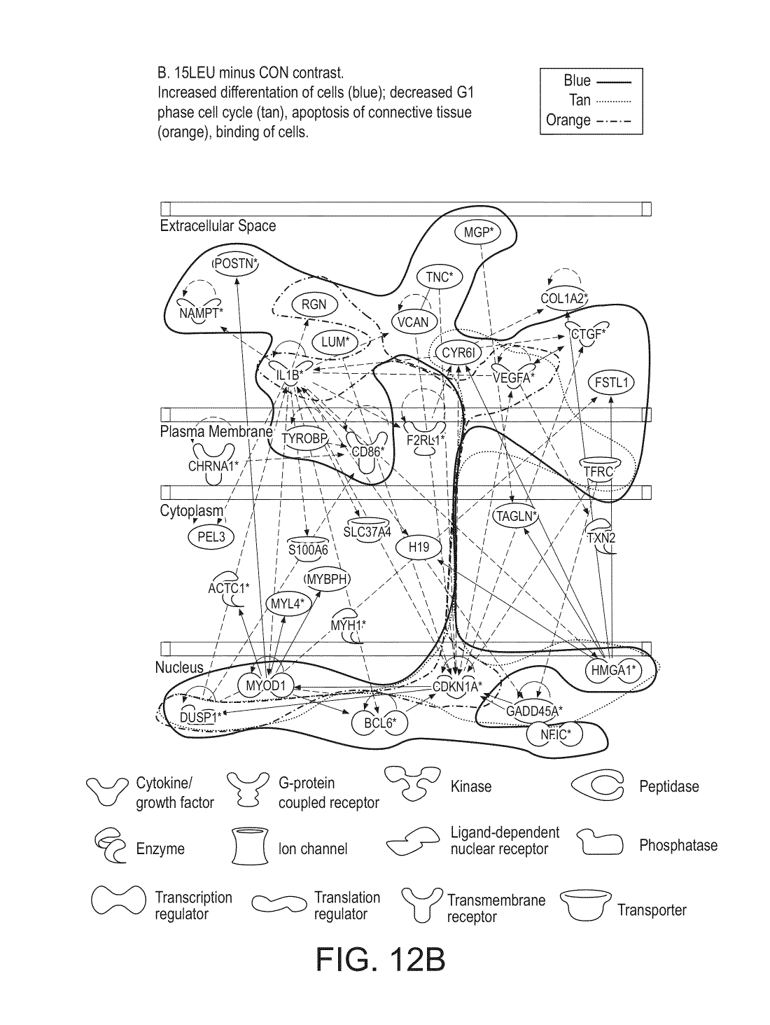

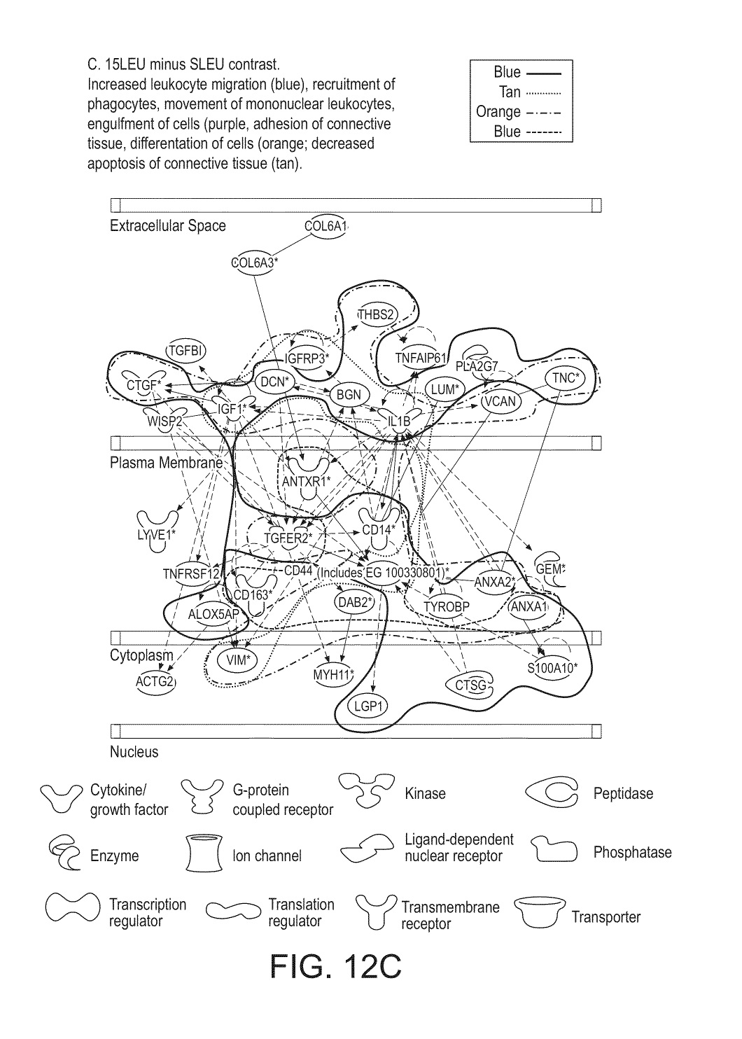

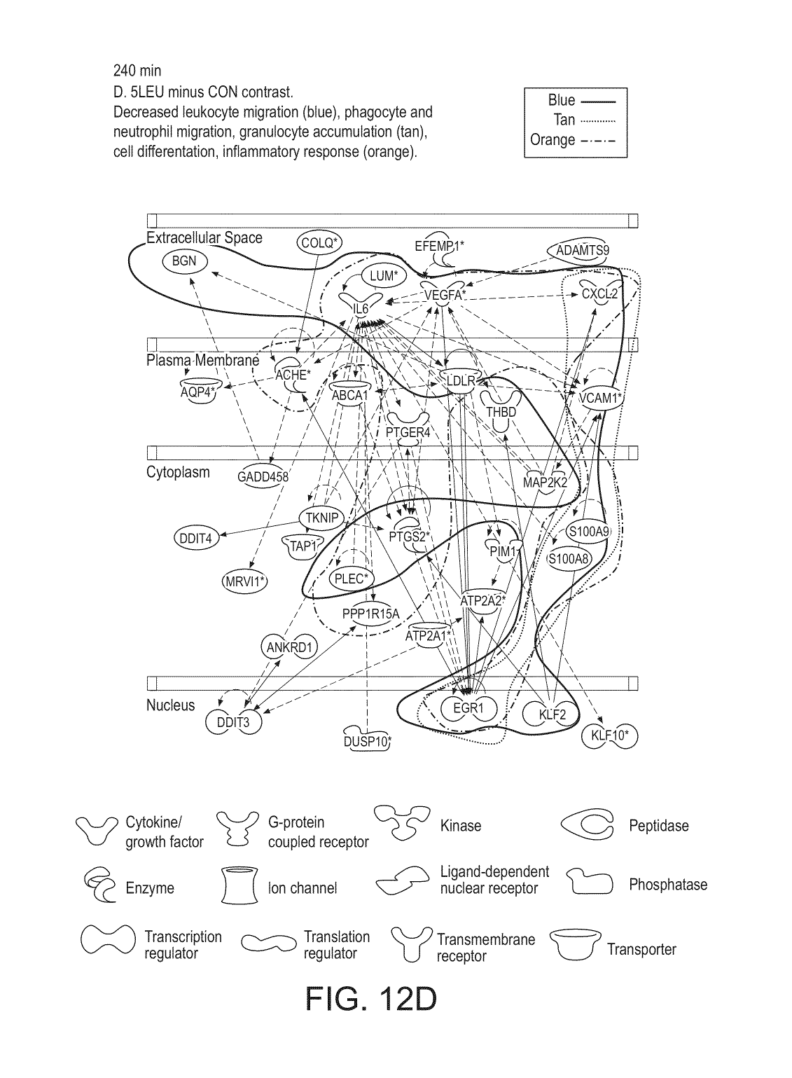

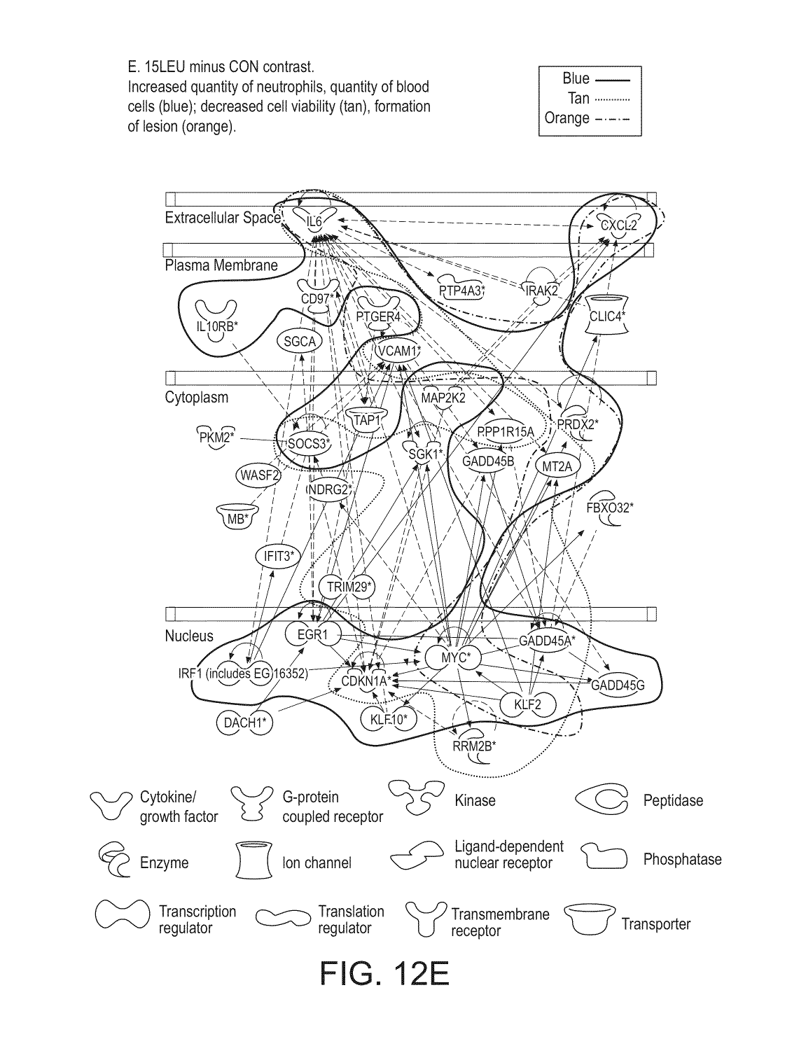

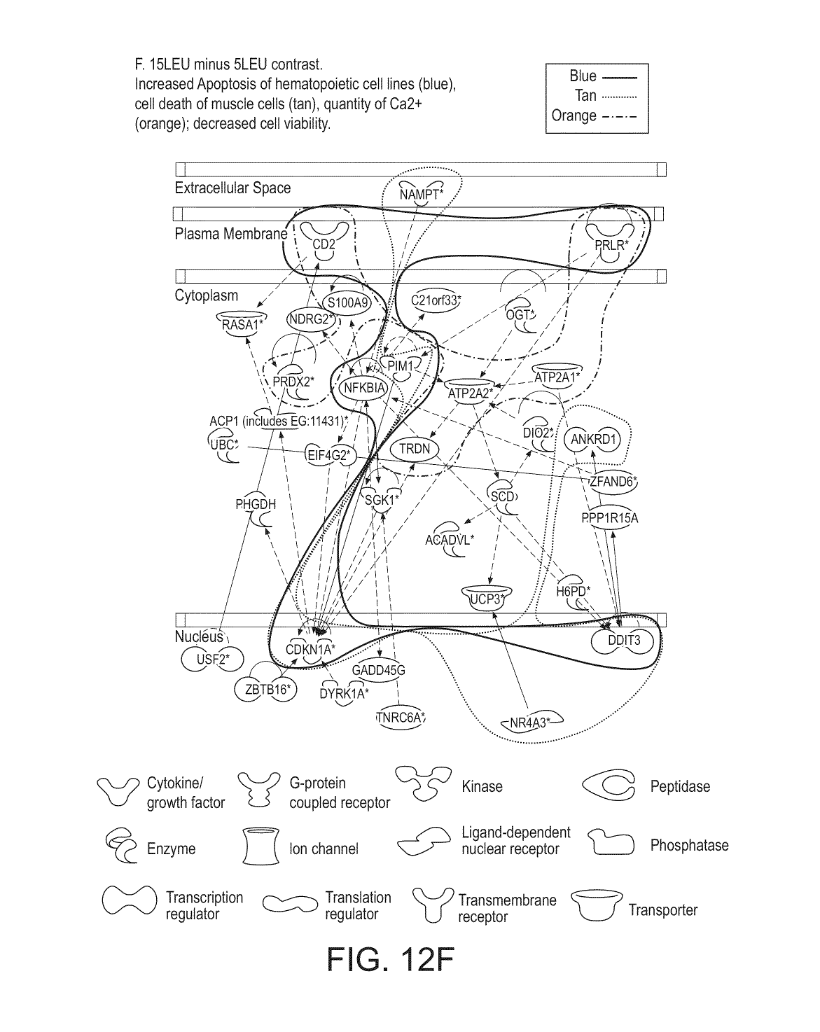

The most resounding functional transcriptome at 30 minutes in response to a single unit of supplement was upregulated immune cell trafficking with the 15LEU-5LEU dose contrast. See, FIG. 12. Geneset interrogation also generated functional modules showing increased cell stability and initiated processes involved in skeletal muscle growth, organization, function and development with both 5LEU and 15LEU doses, relative to CON. See, FIG. 12. By 240 minutes, network analysis indicated a change in the transcriptome programme to progression through a myeloid-cell associated regeneration response, represented by decreased immune-cell migration and growth, anti-inflammatory networks, and increased immune and muscle cell apoptosis with both 5LEU and 15LEU doses, relative to CON. See, FIG. 12.

Functional Connectivity

Hubs tend to encode genes essential for normal cellular function (see, Jeong H, Mason S P, Barabasi A L & Oltvai Z N. (2001). Lethality and centrality in protein networks. Nature 411, 41-42), providing the central network topology to guide functional biological inference inside and between connected modules within the network topology. Accordingly, the central hubs regulating increased leukocyte migration at 30 minutes with 15LEU vs 5LEU were IL1β and CD44. These hubs connected immune-cell differentiation and connective tissue remodeling factors to construct a cell-growth regulatory network that included IGF1 and IGFBP3, TGFB1, and TGFBR2, ECM function, remodeling, adhesion genes (e.g. DCN, BGN, VCAN, TNC, LUM, CTGF), and others involved in macrophage activation and adhesion (CD86, CD44, CD163, CD14, CD68). See, FIG. 12. Additional exploration of immune-cell trafficking networks at 30 minutes revealed expression consistent with a stimulatory impulse for migration, infiltration, adhesion and activation of mononucleocytes, neutrophils, and macrophages. Meanwhile, in the 15LEU vs CON and 5LEU vs CON contrasts at 30 minutes, modular hub gene regulation was consistent with myogenic or satellite cell activation (MYOD1), cell cycle control consistent with cell cycle arrest and increased cell stability via cyclin dependent kinase CDKN1A, GADD45A, and DUSP1. See, FIG. 12.

By 240 minutes, IL6 was the top gene hub. See, FIG. 12. Network connections and the functions analysis suggested progression to an anti-inflammatory expression environment and a change in leukocyte function to increased apoptosis and decreased cell viability that included phagocyte adhesion and ROS production, and decreased expression of connective tissue genes. Down regulation of cell cycle regulators supported progression through apoptosis and differentiation (e.g. GADD45 family genes GADD45A/B/G, CDKN1A; MYC). Metabolic gene expression involving increased metabolism of lipids and nucleic acid components, and the synthesis of steroids, was consistent with cell differentiation.

Upstream Regulators

At 30 minutes, noteworthy in the 15LEU-5LEU contrast was activation of pro-inflammatory cytokines IL1β, SPP1, CCL5, cell cycle arrest and growth regulators (CEBPA/B, EGF), NFKBIA, and components of the SMAD signaling pathway. This outcome was consistent with an early-phase transitory inflammatory response revealed from the network analysis.

By 240 minutes, predicted relative inhibition of the IL6 and IL1β associated inflammatory response occurred in the 5LEU vs CON and 15LEU vs CON contrasts. Furthermore, dose-dependent inhibition of the SMAD signaling pathway (15LEU only), NFKB-RELA, and STAT3 (both 5LEU and 15LEU) suggests reduced cell proliferation, organization of energy metabolism networks, and increased myocellular growth and differentiation with protein-leucine feeding. See, Trenerry M K, Carey K A, Ward A C & Cameron-Smith D. (2007). STAT3 signaling is activated in human skeletal muscle following acute resistance exercise. J Appl Physiol 102, 1483-1489; and Mauro C, Leow S C, Anso E, Rocha S, Thotakura A K, Tornatore L, Moretti M, De Smaele E, Beg A A, Tergaonkar V, Chandel N S & Franzoso G. (2011). NF-kappaB controls energy homeostasis and metabolic adaptation by upregulating mitochondrial respiration. Nature Cell Biol 13, 1272-1279.

Confirmatory Gene Expression

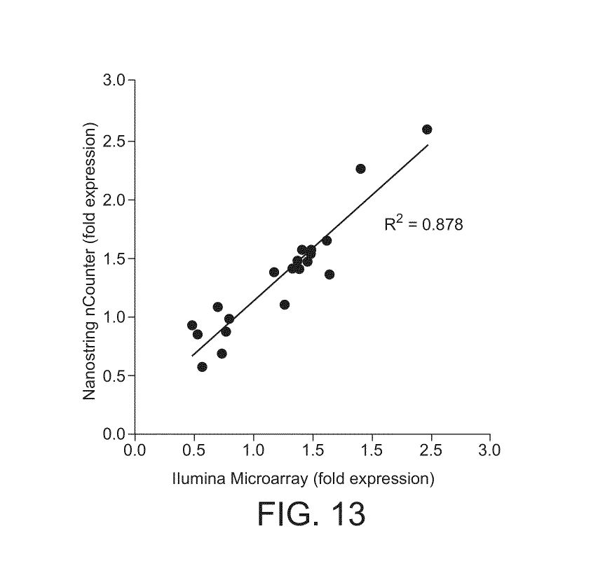

Mean differential gene expression estimated from the microarray was highly correlated (r=0.94) with expression measured by Nanostring. See, FIG. 13.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for enhancing muscle protein synthesis or muscle anabolism in an individual in need of same, the method comprising the steps of:
administering to the individual a therapeutically effective amount of a mixed macronutrient composition having (i) an amount of whey protein that is not capable of enhancing myofibrillar protein synthesis when ingested by itself and (ii) free leucine, wherein the composition comprises at least about 5.0 g total leucine per dose, wherein the composition is administered following a resistance exercise.

2. The method according to claim 1, wherein the amount of whey protein is at least about 6.25 g per dose.

3. The method according to claim 1, wherein the amount of free leucine is at least about 4.25 g per dose.

4. The method according to claim 1, wherein the mixed macronutrient includes a source of carbohydrates and a source of fats.

5. The method according to claim 1, wherein the dose is a single bolus.

6. The method according to claim 1, wherein the composition is administered immediately following a resistance exercise.

7. The method according to claim 1, wherein the composition further comprises at least one of:
at least one source of ω-3 fatty acids selected from the group consisting of fish oil, krill, plant sources containing ω-3 fatty acids, flaxseed, walnut, algae, and combinations thereof;
at least one nucleotide selected from the group consisting of a subunit of deoxyribonucleic acid (“DNA”), a subunit of ribonucleic acid (“RNA”), polymeric forms of DNA and RNA, yeast RNA, and combinations thereof;
at least one phytonutrient selected from the group consisting of flavanoids, allied phenolic compounds, polyphenolic compounds, terpenoids, alkaloids, sulphur-containing compounds, and combinations thereof;
at least one prebiotic selected from the group consisting of acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, and combinations thereof;
at least one probiotic selected from the group consisting of Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, and combinations thereof;
at least one antioxidant selected from the group consisting of astaxanthin, carotenoids, coenzyme Q10 (“CoQ10”), flavonoids, glutathione, Goji (wolfberry), hesperidin, lactowolfberry, lignan, lutein, lycopene, polyphenols, selenium, vitamin A, vitamin C, vitamin E, zeaxanthin, and combinations thereof;
at least one vitamin selected from the group consisting of vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, K1 and K2 (i.e., MK-4, MK-7), folic acid, biotin, and combinations thereof;
at least one mineral selected from the group consisting of boron, calcium, chromium, copper, iodine, iron, magnesium, manganese, molybdenum, nickel, phosphorus, potassium, selenium, silicon, tin, vanadium, zinc, and combinations thereof; or combinations thereof.

8. The method according to claim 1, wherein the composition is a liquid.

9. The method according to claim 1, wherein the nutritional composition is a tube feeding.

10. The method according to claim 1, wherein the nutritional composition is a source of complete nutrition.

11. The method according to claim 1, wherein the nutritional composition is a source of incomplete nutrition.

12. The method according to claim 1, wherein the individual is an athlete.

13. The method according to claim 1, wherein the individual is undergoing muscle rehabilitation.

14. The method according to claim 1, wherein the individual is undergoing physical therapy.

15. The method according to claim 1, wherein the individual is an elderly individual.

16. The method according to claim 1, wherein the individual is a young adult.

17. The method according to claim 1, wherein the nutritional composition is formulated for administration to an individual selected from one of an infant, a child, a young adult, an elderly adult, an athlete, and combinations thereof.
